(19) United States
(12) Patent Application Publication
(10) Pub. No.: US 2019/0189262 A1
(43) Pub. Date: Jun. 20, 2019

(54) SYSTEM AND METHOD FOR FACILITATING ADDICTION RECOVERY

(71) Applicant: PALA-LINQ SOCIAL PURPOSE CORPORATION, Seattle, WA (US)

(72) Inventors: Daniela L. TUDOR, Seattle, WA (US); Chungwoon HAN, Seattle, WA (US); Murphy JENSEN, Poulsbo, WA (US); Jennifer MALLORY, Seattle, WA (US)

(21) Appl. No.: 16/301,041

(22) PCT Filed: May 12, 2017
(86) PCT No.: PCT/US17/32492

§ 371 Date: Nov. 13, 2018

Related U.S. Application Data
(60) Provisional application No. 62/336,113, filed on May 13, 2016.

(51) Int. Cl. G16H 20/70; G16H 40/63

(52) U.S. Cl. CPC G16H 20/70; G16H 40/63

(57) ABSTRACT

A system that supports addiction recovery of individuals includes a number of supporter personal communication devices, a number of individual personal communication devices, at least one communications network, and at least one recovery server. The recovery server can have at least one database stored on respective non-transitory computer- or processor-readable media. The recovery server permits interaction between the supporter personal communication devices and individual personal communication devices while maintaining accountability of entities logically associated with such devices and while addressing a plurality of technical problems that one of ordinary skill in the art can face when implementing the system that supports addiction recovery.

### BACKGROUND

### Technical Field

The present disclosure pertains to addiction recovery and, more particularly, to receiving and real time processing of individual data and implementation of a communication protocol to provide support, user accountability, and inhibit relapse.

### Description of the Related Art

Every year, over 23 million Americans struggle with substance abuse. Additionally, over 23 million are reported to be in recovery from addiction. Addiction tends to isolate the individual from their family and the community, creating unhealthy dependencies and co-dependencies. Recovery from addiction is a complex, self-directed and individualized process that involves deeply emotional and physical experiences.

### BRIEF SUMMARY

The present disclosure is directed to an on-the-go mobile application and related system with wearable and Web components that provide support to those recovering from a variety of addictions, including without limitation, alcohol or drug addiction. By tracking mind, body and spiritual activity levels, the system and method of the present disclosure utilizes a unique technology and protocol that will keep individuals close to their support network and on track with recovery goals to prevent relapse.

In his popular publications, journalist and advocate to end the war on drugs, Johann Hari, concludes that the opposite of addiction is connection. The implementations of the present disclosure will help do just that—leverage technology to keep users connected to their support system, their goals and their overall recovery plan through promoting holistic recovery principles with complementary technology.

The advantages of the device, system, and method of the present disclosure are numerous. Among them are improving performance and minimum requirements of one or more supporter personal communication devices, individual personal communication devices, recovery server, and communications network.

A method may be summarized as including receiving, by at least one recovery server, personal data of an individual, the individual having at least one addiction to at least one activity, behavior, item, or substance; receiving, by the at least one recovery server, at least one meeting indicator, the at least one meeting indicator indicative of at least one meeting to attend by the individual, the at least one meeting including at least one support meeting, group session, or one-on-one session; receiving, by the at least one recovery server, at least one clarity indicator, the at least one clarity indicator indicative of at least one clarity in which to at least one of attend, engage, or participate by the individual, the at least one clarity including at least one spiritual clarity, mental clarity, or physical clarity; receiving, by the at least one recovery server, at least one support contact indicator, the at least one support contact indicator indicative of at least one identifier or contact information of at least one supporter of the individual; creating, by the at least one recovery server, at least one individual profile of the individual based at least in part on at least one of the individual's personal data, the at least one meeting indicator, the at least one clarity indicator, or the at least one support contact indicator; storing, by the at least one recovery server, the at least one individual profile of the individual in at least one nontransitory computer-readable memory; determining, by the at least one recovery server, at least one progress threshold based at least in part on the at least one individual profile of the individual; receiving, by the at least one recovery server, at least one meeting attendance indicator, the at least one meeting attendance indicator indicative of whether the individual at least one of attends or attended one or more of the at least one meeting of the one or more of the at least one meeting; in response to receiving the at least one meeting attendance indicator, determining, by the at least one recovery server, whether the individual at least one of attends or attended the one or more of the at least one meeting; receiving, by the at least one recovery server, at least one clarity success indicator, the at least one clarity success indicator indicative of whether the individual at least one of attends, participates in, engages in, attended, participated in, or engaged in one or more of the at least one clarity; in response to receiving the at least one clarity success indicator, determining, by the at least one recovery server, whether the individual at least one of attends, participates in, engages in, attended, participated in, or engaged in the one or more of the at least one clarity of the one or more of the at least one clarity; receiving, by the at least one recovery server, at least one supporter communication indicator, the at least one supporter communication indicator indicative of whether the individual at least one of communicates or communicated with one or more of the at least one supporter of the individual; in response to receiving the at least one supporter communication indicator, determining, by the at least one recovery server, whether the individual at least one of communicates or communicated with the one or more of the at least one supporter of the individual; generating, by the at least one recovery server, at least one accountability score of the individual based at least in part on i) whether the individual at least one of attends or attended the one or more of the at least one meeting, ii) whether the individual at least one of attends, participates in, engages in, attended, participated in, or engaged in the one or more of the at least one clarity, and iii) whether the individual at least one of communicates or communicated with the one or more of the at least one supporter of the individual; determining, by the at least one recovery server, whether one or more of the accountability score of the individual at least one of equals or exceeds one or more of the at least one progress threshold; in response to the one or more of the at least one accountability score of the individual at least one of equaling or exceeding the one or more of the at least one progress threshold, providing, by the at least one recovery server, at least one progress success indicator to at least one respective personal communication device of at least one of the individual or the at least one supporter of the individual, each of the at least one respective personal communication devices having at least one respective display, the at least one progress success indicator causing the at least one respective personal communication device to display via the at least one respective display at least one progress success notification that indicates to at least one of the individual or the at least one supporter of the individual that the one or more of the at least one accountability score of the individual at least one of equals or exceeds the one or more of the at least one progress threshold; and in response to the one or more of the at least one accountability score of the individual failing to at least one of equal or exceed the one or more of the at least one progress threshold, providing, by the at least one recovery server, at least one progress support indicator to the at least one respective personal communication device of at least one of the individual or the at least one supporter of the individual, the at least one progress support indicator causing the at least one respective personal communication device to display via the at least one respective display at least one progress support alert that alerts at least one of the individual or the at least one supporter of the individual that the one or more of the at least one accountability score of the individual fails to at least one of equal or exceed the one or more of the at least one progress threshold.

Receiving an individual's personal data may further include receiving, by the at least one recovery server, at least one sobriety birthday indicator, the at least one sobriety birthday indicator indicative of at least one sobriety birthday of the individual, the at least one sobriety birthday defined by at least one of i) at least one respective date that the individual most recently engaged or participated in at least one activity or behavior, or ii) at least one respective date that the individual first began a current duration of abstinence from the at least one activity or behavior, the at least one activity or behavior being at least one to which the at least one individual has at least one addiction; and receiving, by the at least one recovery server, at least one support routine indicator, the at least one support routine indicator indicative of at least one course of treatment of addiction of the individual to at least one activity, behavior, substance, or item, the at least one course of treatment including routine attendance of the at least one meeting.

The method may further include determining, by the at least one recovery server, at least one scheduled respective date, respective time, and respective location of each of the at least one meeting, wherein receiving at least one meeting attendance indicator may further include receiving, via at least one respective geolocation system that communicatively couples to the at least one respective personal communication device of the individual and by the at least one recovery server, at least one location indicator, the at least one location indicator indicative of at least one estimated geographic location of the at least one respective personal communication device of the individual as estimated by the at least one respective geolocation system that communicatively couples to the at least one respective personal communication device of the individual; and wherein determining whether the individual at least one of attends or attended the one or more of the at least one meeting may further include determining, by the at least one recovery server, whether the at least one estimated geographic location of the at least one respective personal communication device of the individual at least one of coincides or coincided with the at least one respective location of the one or more of the at least one meeting at the at least one respective date and respective time of the one or more of the at least one meeting.

Receiving at least one location indicator may further include determining, by the at least one recovery server, whether the at least one respective geolocation system that communicatively couples to the at least one respective personal communication device of the individual is enabled; and in response to the at least one respective geolocation system that communicatively couples to the at least one respective personal communication device of the individual not being enabled, providing, to the at least one respective personal communication device of the individual and by the at least one recovery server, at least one geolocation enablement communication that causes the at least one respective personal communication device of the individual to display via the respective display at least one geolocation enablement notification that at least one of instructs or prompts the individual to enable the at least one geolocation system that communicatively couples to the at least one respective personal communication device of the individual.

The method may further include determining, by the at least one recovery server, at least one respective clarity biometric value of each of the at least one clarity, the at least one respective clarity biometric value indicative of at least one estimated respective biometric of the individual while the individual at least one of attends, participates in, or engages in each of the at least one clarity, wherein receiving at least one clarity success indicator may further include receiving, via at least one respective biometric sensor that communicatively couples to the at least one respective personal communication device of the individual and by the at least one recovery server, at least one biometric indicator, the at least one biometric indicator indicative of at least one estimated biometric of the individual as estimated by the at least one respective biometric sensor that communicatively couples to the at least one respective personal communication device of the individual; and wherein determining whether the individual at least one of attends, participates in, engages in, attended, participated in, or engaged in the one or more of the at least one clarity may further include determining, by the at least one recovery server, whether the at least one estimated biometric of the individual coincides or coincided with the at least one estimated respective biometric of the individual while the individual attends, participates in, or engages in the one or more of the at least one clarity.

Determining at least one respective clarity biometric value of each of the at least one clarity may further include determining, by the at least one recovery server, the at least one respective clarity biometric value of each of the at least one clarity based at least in part on at least one of i) at least one historical estimated respective biometric of the individual while the individual attends, participates in, or engages in each of the at least one clarity, or ii) at least one portion of the at least one individual profile of the individual. The at least one spiritual clarity may include at least one of prayer, meditation, or religious service, wherein the at least one mental clarity may include at least one of creative exercise, reading, or writing, and wherein the at least one physical clarity may include at least one of physical exercise, hiking, or sleeping.

A system may be summarized as including at least one personal communication device of an individual, the at least one personal communication device of the individual having at least one respective processor, nontransitory computer-readable memory communicatively coupled to the at least one respective processor, display communicatively coupled to the at least one respective processor, and individual input interface communicatively coupled to the at least one respective processor, the individual having at least one addiction to at least one activity, behavior, item, or substance; and at least one recovery server that communicatively couples to the personal communication device of the individual via at least one communications network, the at least one recovery server having at least one processor and nontransitory computer-readable memory communicatively coupled to the at least one processor, wherein one or more of the at least one respective nontransitory computer-readable memory of the at least one personal communication device of the individual or the at least one nontransitory computer-readable memory of the at least one recovery server having at least one of respective processor-executable instructions or data stored therein, the at least one of respective processor-executable instructions or data which, when executed by one or more of the at least one respective processor of the at least one personal communication device or the at least one processor of the at least one recovery server, cause the at least one personal communication device of the individual and the at least one recovery server to cooperatively support recovery of the individual by one or more of the at least one personal communication device of the individual or the at least one recovery server: obtaining personal data of the individual; obtaining at least one of each indicator in a group of indicators that may include two or more selected from a meeting indicator, a clarity indicator, or a support contact indicator, the at least one meeting indicator indicative of at least one meeting to attend by the individual, the at least one meeting including at least one support meeting, group session, or one-on-one session, the at least one clarity indicator indicative of at least one clarity in which to at least one of attend, engage, or participate by the individual, the at least one clarity including at least one spiritual clarity, mental clarity, or physical clarity, the at least one support contact indicator indicative of at least one identifier or contact information of at least one supporter of the individual; creating at least one individual profile of the individual based at least in part on at least one of the individual's personal data, the at least one meeting indicator, the at least one clarity indicator, or the at least one support contact indicator; storing the at least one individual profile of the individual in one or more of the at least one respective nontransitory computer-readable memory of the at least one personal communication device or the at least one nontransitory computer-readable memory of the at least one recovery server; determining at least one progress threshold based at least in part on the at least one individual profile of the individual; obtaining at least one of each respective indicator in an indicator group of indicators that may include two or more selected from meeting attendance indicator, clarity success indicator, or supporter communication indicator, the at least one meeting attendance indicator indicative of whether the individual at least one of attends or attended one or more of the at least one meeting of the one or more of the at least one meeting, the at least one clarity success indicator indicative of whether the individual at least one of attends, participates in, engages in, attended, participated in, or engaged in one or more of the at least one clarity, the at least one supporter communication indicator indicative of whether the individual at least one of communicates or communicated with one or more of the at least one supporter of the individual; in response to obtaining the at least one meeting attendance indicator, determining whether the individual at least one of attends or attended the one or more of the at least one meeting; in response to obtaining the at least one clarity success indicator, determining whether the individual at least one of attends, participates in, engages in, attended, participated in, or engaged in the one or more of the at least one clarity of the one or more of the at least one clarity; in response to obtaining the at least one supporter communication indicator, determining whether the individual at least one of communicates or communicated with the one or more of the at least one supporter of the individual; generating at least one accountability score of the individual based at least in part on at least two of i) whether the individual at least one of attends or attended the one or more of the at least one meeting, ii) whether the individual at least one of attends, participates in, engages in, attended, participated in, or engaged in the one or more of the at least one clarity, or iii) whether the individual at least one of communicates or communicated with the one or more of the at least one supporter of the individual; determining whether one or more of the accountability score of the individual at least one of equals or exceeds one or more of the at least one progress threshold; in response to the one or more of the at least one accountability score of the individual at least one of equaling or exceeding the one or more of the at least one progress threshold, providing at least one progress success indicator to one or more of the at least one personal communication device of the individual or at one personal communication device of the at least one supporter of the individual, the at least one personal communication device of the at least one supporter of the individual having at least one respective display, the at least one progress success indicator causes the one or more of the at least one personal communication device of the individual or the at least one personal communication device of the at least one supporter of the individual to display via the at least one respective display of the one or more of the at least one personal communication device of the individual or the at least one personal communication device of the at least one supporter of the individual at least one progress success notification that indicates to at least one of the individual or the at least one supporter of the individual that the one or more of the at least one accountability score of the individual at least one of equals or exceeds the one or more of the at least one progress threshold; and in response to the one or more of the at least one accountability score of the individual failing to at least one of equal or exceed the one or more of the at least one progress threshold, providing at least one progress support indicator to the one or more of the at least one personal communication device of the individual or the at least one personal communication device of the at least one supporter of the individual, the at least one progress support indicator causing the one or more of the at least one personal communication device of the individual or the at least one personal communication device of the at least one supporter of the individual to display via the at least one respective display of the one or more of the at least one personal communication device of the individual or the at least one personal communication device of the at least one supporter of the individual at least one progress support alert that alerts at least one of the individual or the at least one supporter of the individual that the one or more of the at least one accountability score of the individual fails to at least one of equal or exceed the one or more of the at least one progress threshold.

Obtaining an individual's personal data may further include obtaining at least one sobriety birthday indicator, the at least one sobriety birthday indicator indicative of at least one sobriety birthday of the individual, the at least one sobriety birthday defined by at least one of i) at least one respective date that the individual most recently engaged or participated in at least one activity or behavior, or ii) at least one respective date that the individual first began a current duration of abstinence from the at least one activity or behavior, the at least one activity or behavior being at least one to which the at least one individual has at least one addiction; and obtaining at least one support routine indicator, the at least one support routine indicator indicative of at least one course of treatment of addiction of the individual to at least one activity, behavior, substance, or item, the at least one course of treatment including routine attendance of the at least one meeting.

The at least one of respective processor-executable instructions or data which, when executed by one or more of the at least one respective processor of the at least one personal communication device of the individual or the at least one processor of the at least one recovery server, may further cause the at least one personal communication device of the individual and the at least one recovery server to cooperatively support recovery of the individual by one or more of the at least one personal communication device of the individual or the at least one recovery server: determining at least one scheduled respective date, respective time, and respective location of each of the at least one meeting, wherein the indicator group may include the at least one meeting indicator, wherein obtaining at least one of each respective indicator in an indicator group of indicators that may include two or more selected from meeting attendance indicator, clarity success indicator, or supporter communication indicator may further include obtaining, via at least one respective geolocation system that communicatively couples to the at least one personal communication device of the individual, at least one location indicator, the at least one location indicator indicative of at least one estimated geographic location of the at least one personal communication device of the individual as estimated by the at least one respective geolocation system that communicatively couples to the at least one personal communication device of the individual; and wherein determining whether the individual at least one of attends or attended the one or more of the at least one meeting may further include determining whether the at least one estimated geographic location of the at least one personal communication device of the individual at least one of coincides or coincided with the at least one respective location of the one or more of the at least one meeting at the at least one respective date and respective time of the one or more of the at least one meeting.

Obtaining at least one location indicator may further include determining whether the at least one respective geolocation system that communicatively couples to the at least one personal communication device of the individual is enabled; and in response to the at least one respective geolocation system that communicatively couples to the at least one personal communication device of the individual not being enabled, displaying, via the respective display of the at least one personal communication device of the individual, at least one geolocation enablement notification that at least one of instructs or prompts the individual to enable the at least one geolocation system that communicatively couples to the at least one personal communication device of the individual.

The at least one of respective processor-executable instructions or data which, when executed by one or more of the at least one respective processor of the at least one personal communication device of the individual or the at least one processor of the at least one recovery server, may further cause the at least one personal communication device of the individual and the at least one recovery server to cooperatively support recovery of the individual by one or more of the at least one personal communication device of the individual or the at least one recovery server: determining at least one respective clarity biometric value of each of the at least one clarity, the at least one respective clarity biometric value indicative of at least one estimated respective biometric of the individual while the individual at least one of attends, participates in, or engages in each of the at least one clarity, wherein the indicator group may include the at least one clarity indicator, wherein obtaining at least one clarity success indicator may further include obtaining, via at least one respective biometric sensor that communicatively couples to the at least one personal communication device of the individual, at least one biometric indicator, the at least one biometric indicator indicative of at least one estimated biometric of the individual as estimated by the at least one respective biometric sensor that communicatively couples to the at least one personal communication device of the individual; and wherein determining whether the individual at least one of attends, participates in, engages in, attended, participated in, or engaged in the one or more of the at least one clarity may further include determining whether the at least one estimated biometric of the individual coincides or coincided with the at least one estimated respective biometric of the individual while the individual attends, participates in, or engages in the one or more of the at least one clarity.

Determining at least one respective clarity biometric value of each of the at least one clarity may further include determining the at least one respective clarity biometric value of each of the at least one clarity based at least in part on at least one of i) at least one historical estimated respective biometric of the individual while the individual attends, participates in, or engages in each of the at least one clarity, or ii) at least one portion of the at least one individual profile of the individual.

The indicator group may include the at least one support contact indicator, and determining whether the individual at least one of communicates or communicated with the one or more of the at least one supporter of the individual may further include analyzing at least one telephone call, text message, or email that the at least one personal communication device of the individual at least one of transmits or receives; and determining whether the at least one analyzed telephone call, text message, or email includes the at least one supporter of the individual as at least one recipient or sender of the at least one analyzed telephone call, text message, or email.

The indicator group may include the at least one support contact indicator, and determining whether the individual at least one of communicates or communicated with the one or more of the at least one supporter of the individual may further include obtaining, via at least one respective geolocation system that communicatively couples to the at least one personal communication device of the individual, at least one individual location indicator, the at least one individual location indicator indicative of at least one estimated individual geographic location of the at least one personal communication device of the individual as estimated by the at least one respective geolocation system that communicatively couples to the at least one personal communication device of the individual; obtaining, via at least one respective geolocation system that communicatively couples to the at least one personal communication device of the at least one supporter of the individual, at least one supporter location indicator, the at least one supporter location indicator indicative of at least one estimated supporter geographic location of the at least one personal communication device of the at least one supporter of the individual as estimated by the at least one respective geolocation system that communicatively couples to the at least one personal communication device of the at least one supporter of the individual; and determining whether the at least one estimated individual geographic location of the at least one personal communication device of the individual at least one of coincides or coincided with the at least one estimated supporter geographic location of the at least one personal communication device of the at least one supporter of the individual.

The indicator group may include the at least one support contact indicator, and determining whether the individual at least one of communicates or communicated with the one or more of the at least one supporter of the individual may further include obtaining, via at least one respective near field communication (NFC) system or personal area network (PAN) system that communicatively couples to the at least one personal communication device of the individual, at least one NFC or PAN communication indicator, the at least one NFC or PAN communication indicator indicative of at least one identifier of at least one NFC system, PAN system, or personal communication device of another to which the NFC or PAN system that communicatively couples to the at least one personal communication device of the individual communicatively couples; and determining whether the at least one identifier of the at least one NFC system, PAN system, or personal communication device of the other matches at least one identifier of at least one NFC system, PAN system, or personal communication device of the at least one supporter of the individual.

The at least one of respective processor-executable instructions or data which, when executed by one or more of the at least one respective processor of the at least one personal communication device of the individual or the at least one processor of the at least one recovery server, may further cause the at least one personal communication device of the individual and the at least one recovery server to cooperatively support recovery of the individual by one or more of the at least one personal communication device of the individual or the at least one recovery server: intermittently or continuously adjusting the one or more of the at least one progress threshold based at least in part on at least one of a number or percentage of scheduled activities of the individual that the individual successfully achieves in a defined period of time that includes multiple scheduled activities.

The at least one of respective processor-executable instructions or data which, when executed by one or more of the at least one respective processor of the at least one personal communication device of the individual or the at least one processor of the at least one recovery server, may further cause the at least one personal communication device of the individual and the at least one recovery server to cooperatively support recovery of the individual by one or more of the at least one personal communication device of the individual or the at least one recovery server: obtaining, via at least one respective biometric sensor that communicatively couples to the at least one personal communication device of the individual, at least one biometric indicator, the at least one biometric indicator indicative of at least one estimated biometric of the individual as estimated by the at least one respective biometric sensor that communicatively couples to the at least one personal communication device of the individual; determining at least one stress level that the at least one estimated biometric of the individual indicates; and intermittently or continuously adjusting at least one of respective frequency or respective duration of at least one scheduled activity of the individual responsive to the stress level.

### BRIEF DESCRIPTION OF THE SEVERAL VIEWS OF THE DRAWINGS

The foregoing and other features and advantages of the present disclosure will be more readily appreciated as the same become better understood from the following detailed description when taken in conjunction with the accompanying drawings.

In the drawings, identical reference numbers identify similar elements or acts. The sizes and relative positions of elements in the drawings are not necessarily drawn to scale. For example, the shapes of various elements and angles are not necessarily drawn to scale, and some of these elements can be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings.

FIG. 1 is a schematic view of an environment in which a system supports recovery of individuals that have at least one respective addition to at least one respective activity, behavior, item, or substance according to at least one illustrated implementation, the system including devices logically associated with respective supporters of the individuals, devices logically associated with the respective individuals, at least one communications network, and at least one recovery server that communicably couples to at least one of the devices logically associated with respective ones of the supporters via the communications network and at least one of the devices logically associated with respective ones of the individuals via the communications network.

FIG. 2 is a block diagram of the system that supports recovery of FIG. 1 according to at least one illustrated implementation, better illustrating various components of the recovery server of FIG. 1, supporter personal communication devices logically associated with respective supporters, and individual personal communication devices logically associated with respective individuals, all communicatively coupled by the communications network.

FIGS. 3-33 are screen shots of at least one individual graphical interface that an individual personal communication device of FIG. 1 can display, according to at least one illustrated implementation.

FIG. 34 is a screen shot of at least one supporter graphical interface that a supporter personal communication device of FIG. 1 can display, according to at least one illustrated implementation.

### DETAILED DESCRIPTION

In the following description, certain specific details are set forth in order to provide a thorough understanding of various disclosed implementations. However, one skilled in the relevant art will recognize that implementations can be practiced without one or more of these specific details, or with other methods, components, materials, etc. In other instances, well-known structures or components or both associated with computers, cell phones, and personal devices as well as Global Positioning System, computer networks, and the Internet have not been shown or described in order to avoid unnecessarily obscuring descriptions of the implementations.

Unless the context requires otherwise, throughout the specification and claims that follow, the word “comprise” and variations thereof, such as “comprises” and “comprising,” are to be construed in an open inclusive sense, that is, as “including, but not limited to.” The foregoing applies equally to the words “including” and “having.”

Reference throughout this description to “one implementation” or “an implementation” means that a particular feature, structure, or characteristic described in connection with the implementation is included in at least one implementation. Thus, the appearance of the phrases “in one implementation” or “in an implementation” in various places throughout the specification are not necessarily all referring to the same implementation. Furthermore, the particular features, structures, or characteristics can be combined in any suitable manner in one or more implementations.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

The headings and Abstract of the Disclosure provided herein are for convenience only and do not interpret the scope or meaning of the implementations.

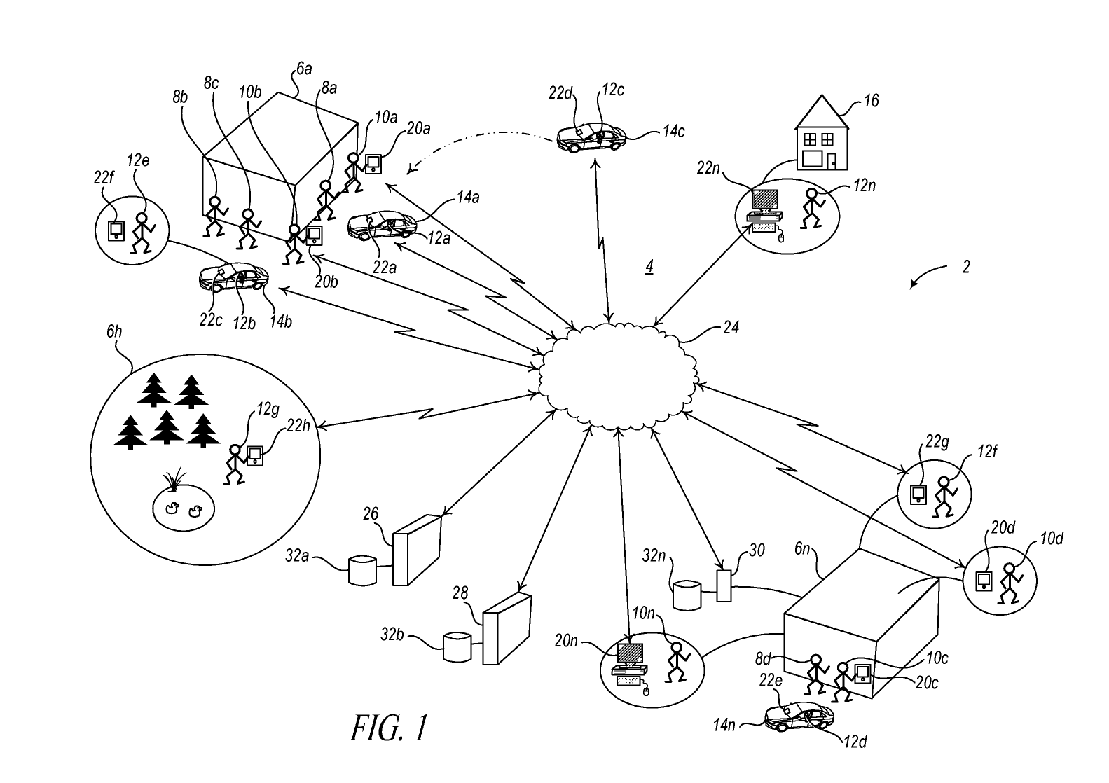

FIG. 1 shows an environment 4 in which a system 2 supports recovery, according to at least one illustrated implementation.

The environment 4 can include a number of facilities 6a (collectively 6a; only one shown), a number of non-connected individuals 8a-8n (collectively 8; only four shown), a number of supporters 10a-10n (collectively 10; only five shown), a number of connected individuals 12a-12n (collectively 12; only eight shown), a number of vehicles 14a-14n (collectively 14; only four shown), and a residence 16 (only one shown). The supporters 10 can include friends, counselors, case managers, parole officers, therapists, parents, or other individuals 12.

A first facility 6a can be an in-patient rehabilitation treatment center. A second facility 6b can be a public park. A third facility can host at least one addiction recovery meeting.

The individuals 8, 12 have at least one respective addiction to at least one respective activity, behavior, item, or substance. For example, a given one of the individuals 8, 12 may have at least one addiction to at least one drug, alcohol, sex, gambling, theft, technology, lying, gaming, an eating disorder, food, exercise, etc.

The system 2 comprises at least one personal communication device 22a-22n (collectively 22; only eight shown) of at least a respective one of the individuals 12. The system 2 can also comprise at least one personal communication device 20a-20n (collectively 20; only five shown) of at least a respective one of the supporters 10. The system 2 also comprises at least one recovery server 28.

The supporter personal communication devices 20 can, for example, take the form of any variety of communications enabled devices, including wireless communications devices such as tablet computers, smartphones, dudphones (e.g., a phone that, as opposed to a smartphone, permits limited operation such as only one or more operation explained herein), netbook computers with wireless modems 262, wireless enabled headunits of vehicles, wearable devices (e.g., bracelet, anklet, watch, necklace, glasses, headband, in-ear device), and implantable devices (e.g., subcutaneous implanted device). The individual personal communication devices 22 can, for example, take the form of any variety of communications enabled devices, including wireless communications devices such as tablet computers, smartphones, dudphones, netbook computers with wireless modems 262, wireless enabled headunits of vehicles, wearable devices (e.g., bracelet, anklet, watch, necklace, glasses, headband, in-ear device), and implantable devices (e.g., subcutaneous implanted device). The supporter personal communication devices 20 or the individual personal communication devices 22, or both can, for example, each respectively store one or more unique identifiers, for example, a Mobile Identification Number (MIN), Mobile Subscription Identification Number (MSIN), Mobile Station Identifier (MSID), International Mobile Subscriber Identity (IMSI), or International Mobile Station Equipment Identity (IMEI). Such can be stored in the device in a non-removable memory or in a removable memory (e.g., Subscriber Identity Module (SIM) card).

Each of the supporter personal communication devices 20, individual personal communication devices 22, or recovery server 28 communicably couple to the communications network 24 via at least one respective wired connection (e.g., electrically conductive wires, optical fiber) or at least one respective wireless connection (e.g., radio, WI-FI® radio, Bluetooth® radio, cellular radio, optical emitter and sensor pair such as an infrared emitter and sensor pair). The recovery server 28 can communicably couple to at least one of the supporter personal communication devices 20 or individual personal communication devices 22 via the communications network 24.

The recovery server 28 logically associates in the database stored on one of the nontransitory processor-readable media 32b, 32n at least one of the supporter personal communication devices 20 with at least one of the facilities 6. Additionally or alternatively, the recovery server 28 can logically associate in the database stored on the nontransitory processor-readable media 32b at least one of the supporter personal communication devices 20 with at least one of the supporters 10, for example, logically associating a unique identifier of a mobile communications device (e.g., smartphone) with a name or identifier that uniquely identifies the supporter 10. For example, each of the supporter personal communication devices 20, facilities 6, or supporters 10 can have at least one respective unique identifier (e.g., globally unique, locally unique to at least one of the facilities 6).

The recovery server 28 logically associates in the database stored on one of the nontransitory processor-readable media 32b at least one of the individual personal communication devices 22 with at least one of the individuals 12, for example, logically associating a unique identifier of a mobile communications device (e.g., smartphone or dudphone) with a name or identifier that uniquely identifies the individuals 12. In other words, each of the individual personal communication devices 22, or individuals 12 can have at least one respective unique identifier (e.g., globally unique, locally unique to at least one of the facilities 6).

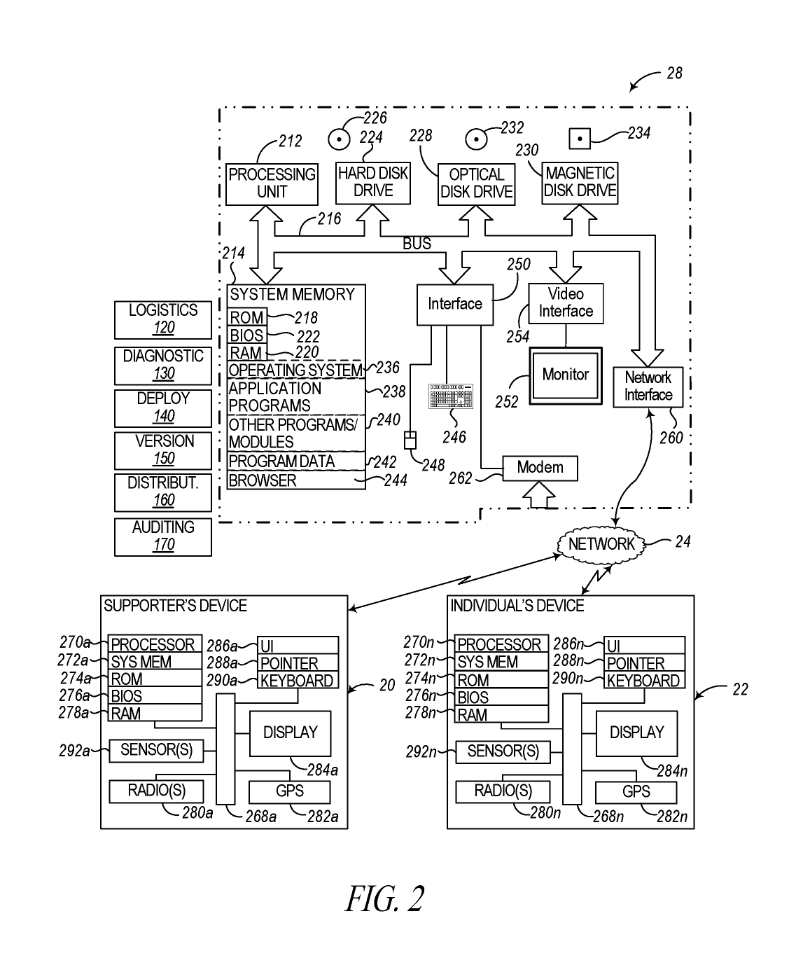

FIG. 2 and the following discussion provide a brief, general description of an example suitable computing environment of the system 2 of FIG. 1. The example suitable computing environment includes the recovery server 28. The recovery server 28 incorporates an Inventory Logistics System 120, a Diagnostics System 130, a Deployment and Positioning System 140, an Instruction Versioning Approval System 150, an Instruction Content Distribution System 160, and an Auditing System 170. The recovery server 28 communicably couples to at least one communications network 24. The communications network 24 communicably couples the recovery server 28 to example ones of the supporter personal communication devices 20 and the individual personal communication devices 22.

Although not required, several implementations are described in the general context of computer-executable instructions, such as program application modules, objects, or macros being executed by a computer. Those skilled in the relevant art will appreciate that the illustrated implementations as well as other implementations can be practiced with other computer system configurations, including hand-held devices, multiprocessor systems, microprocessor-based or programmable consumer electronics, personal computers (“PCs”), network PCs, mini computers, mainframe computers, and the like, specifically programmed or configured to operate according to at least one algorithm explained herein. The implementations can be practiced in distributed computing environments where tasks or modules are performed by remote processing devices, which are linked through the communications network 24. In a distributed computing environment, program modules can be located in both local and remote memory storage devices. For example, anything explained herein with regard to at least one of the supporter personal communication devices 20, individual personal communication devices 22, or recovery server 28 can additionally or alternatively be at least one of performed by or located in at least one component of at least another one of the supporter personal communication devices 20, individual personal communication devices 22, or recovery server 28. In particular, by at least one of the supporter personal communication devices 20, individual personal communication devices 22, or recovery server 28 of the system 2 cooperatively executing at least one portion of at least one of the algorithms explained herein in cooperation with at least another at least one of the supporter personal communication devices 20, individual personal communication devices 22, or recovery server 28 of the system 2, the system 2 alleviates consumption of processing or network resources of at least one of the supporter personal communication devices 20, individual personal communication devices 22, or recovery server 28, thereby improving performance and minimum requirements of at least one of the supporter personal communication devices 20, individual personal communication devices 22, recovery server 28, or communications network 24.

The recovery server 28 can take the form of a mainframe or mini-computer that includes a processing unit 212, an at least partially nontransitory system memory 214 and a system bus 216 that couples various system components including the system memory 214 to the processing unit 212. The recovery server 28 will at times be referred to in the singular herein, but this is not intended to limit the implementations to a single recovery server since in typical implementations, there will be more than one recovery server 28 or other device involved. The computing environment can employ other computers, such as personal computers, where the size or scale of the system allows, specifically programmed or configured to operate according to at least one algorithm explained herein.

The processing unit 212 can be any logic processing unit, such as one or more central processing units (CPUs), digital signal processors (DSPs), application-specific integrated circuits (ASICs), etc., specifically programmed or configured to operate according to at least one algorithm explained herein. The system bus 216 can employ any known bus structures or architectures, including a memory bus with memory controller, a peripheral bus, and a local bus. The system memory 214 includes read-only memory (“ROM”) 218 and random access memory (“RAM”) 220. A basic input/output system (“BIOS”) 222, which can form part of the ROM 218, contains basic routines that help transfer information between elements within the back-end system 28, such as during start-up.

The recovery server 28 also includes a hard disk drive 224 for reading from and writing to a hard disk 226, and an optical disk drive 228 and a magnetic disk drive 230 for reading from and writing to removable optical disks 232 and magnetic disks 234, respectively. The optical disk 232 can be a CD-ROM, while the magnetic disk 234 can be a magnetic floppy disk or diskette. The hard disk drive 224, optical disk drive 228 and magnetic disk drive 230 communicate with the processing unit 212 via the system bus 216. The hard disk drive 224, optical disk drive 228 and magnetic disk drive 230 can include interfaces or controllers (not shown) coupled between such drives and the system bus 216. The drives 224, 228 and 230, and their associated computer-readable media 226, 232, 234, provide nonvolatile storage of computer readable instructions, data structures, program modules and other data for the back-end system 28. Although the depicted back-end system 28 employs hard disk 224, optical disk 228, and magnetic disk 230, those skilled in the relevant art will appreciate that other types of computer-readable media that can store data accessible by a computer can be employed, such as magnetic cassettes, flash memory cards, digital video disks (“DVD”), Bernoulli cartridges, RAMs, ROMs, smart cards, solid state drives, etc.

Program modules can be stored in the system memory 214, such as an operating system 236, one or more application programs 238, other programs or modules 240 and program data 242. The system memory 214 can also include communications programs, for example, a Web client or browser 244 for permitting the back-end system 28 to access and exchange data with sources such as Web sites of the Internet, corporate intranets, or other networks as described below, as well as other server applications on server computing systems such as those discussed further below. The browser 244 in the depicted implementation is markup language based, such as Hypertext Markup Language (HTML), Extensible Markup Language (XML) or Wireless Markup Language (WML), and operates with markup languages that use syntactically delimited characters added to the data of a document to represent the structure of the document. A number of Web clients or browsers are commercially available, such as those from America Online® and Microsoft® of Redmond, Wash.

While shown in FIG. 2 as being stored in the system memory 214, the operating system 236, application programs 238, other programs/modules 240, program data 242 and browser 244 can be stored on the hard disk 226 of the hard disk drive 224, the optical disk 232 of the optical disk drive 228 and/or the magnetic disk 234 of the magnetic disk drive 230. A system operator can enter commands and information into the recovery server 28 through input devices such as a touch screen or keyboard 246 and/or a pointing device such as a mouse 248. Other optional input devices can include a microphone, joystick, game pad, tablet, scanner, etc. These and other input devices are communicably coupled to the processing unit 212 through an interface 250 such as a universal serial bus (“USB”) interface that couples to the system bus 216, although other interfaces such as a parallel port, a game port or a wireless interface or serial port can be used or a network interface 260. A supporter 252 or other display device is coupled to the system bus 216 via a video interface 254, such as a video adapter. The recovery server 28 can include other output devices, such as speakers, printers, etc.

At least one of the supporter personal communication devices 20 or individual personal communication devices 22 can be any logic processing unit, such as one or more central processing units (CPUs), digital signal processors (DSPs), application-specific integrated circuits (ASICs), etc., specifically programmed or configured to operate according to at least one algorithm explained herein. The system bus 268 can employ any known bus structures or architectures, including a memory bus with memory controller, a peripheral bus, and a local bus. The system memory 272 includes read-only memory or memories (“ROM”) 274n and at least one or more random access memory (“RAM”) 278n. A basic input/output system or systems (“BIOS”) 276n, which can form part of the ROM 274n, contains basic routines that help transfer information between elements within the portable device 110, such as during start-up.

At least one of the supporter personal communication devices 20 or individual personal communication devices 22 also respectively include one or more storage drives (not shown). The one or more storage drives ideally contain data indicative of one or more checklists or instruction sets or both. The storage drives can include any type, number, or combination, of non-volatile, nontransitory storage media including magnetic storage media, optical storage media, electromagnetic storage media, molecular or atomic storage media, quantum storage media, or combinations thereof. At least one of the supporter personal communication devices 20 or individual personal communication devices 22 also respectively include one or more processors 270a-270n (collectively 270; only two shown). The storage drive(s) communicate with the processor 270 via the system bus 268. At least one of the supporter personal communication devices 20 or individual personal communication devices 22 also respectively include one or more system buses 268a-268n (collectively 268; only two shown). The one or more storage drives 280 can include interfaces or controllers (not shown) coupled between such drives and the system bus 268, as is known by those skilled in the relevant art. The one or more storage drives provide nonvolatile storage of computer readable instructions, data structures, program modules and other data for at least one of the supporter personal communication devices 20 or individual personal communication devices 22.

At least one of the supporter personal communication devices 20 or individual personal communication devices 22 also respectively include one or more system memory 272a-272n (collectively 272; only two shown). Program modules can be stored in the system memory 272, such as an operating system, one or more application programs, other programs or modules and program data. The system memory 272 can also include communications programs, for example, a Web client or browser for permitting at least one of the supporter personal communication devices 20 or individual personal communication devices 22 to access and exchange data with sources such as Web sites of the Internet, corporate intranets, or other networks as described below, as well as other server applications on server computing systems such as those discussed further below. The browser in the depicted implementation is markup language based, such as Hypertext Markup Language (HTML), Extensible Markup Language (XML) or Wireless Markup Language (WML), and operates with markup languages that use syntactically delimited characters added to the data of a document to represent the structure of the document. A number of Web clients or browsers are commercially available such as those from Google®, Apple®, America Online® and Microsoft® of Redmond, Wash.

At least one of the supporter personal communication devices 20 or individual personal communication devices 22 also respectively include one or more communications interfaces 280a-280n (collectively 280; only two shown). The communications interfaces 280 communicably coupled to the system bus 268. The one or more communications interfaces 280 can include one or more Bluetooth® interfaces, one or more near field communications (“NFC”) interfaces, one or more cellular communications interfaces, one or more radio frequency identification (“RFID”) interfaces, or any other RF or microwave interface capable of supporting a data exchange with an external device such as at least one of the individual personal communication devices 22a, 22c, 22d, or 22e of the vehicles 14. The one or more communications interfaces 280 can also include one or more tethered communications interfaces such as an IEEE 1394 (“Firewire”) interface, a USB interface, or similar standard or proprietary interface capable of supporting a data exchange with an external device such as at least one of the individual personal communication devices 22a, 22c, 22d, or 22e of the vehicles 14. In at least some implementations, any number of communications interfaces 280 can be in simultaneous use by at least one of the supporter personal communication devices 20 or individual personal communication devices 22. For example, a given one of the individual personal communication devices 22a, 22c, 22d, or 22e (e.g., head units) of the vehicles 14 can communicate vehicle identification and position data to at least another one of the individual personal communication devices 22 via, for example, a wireless IEEE 802.11 interface of the communications interfaces 280. At the same time, at least one of the supporter personal communication devices 20 or individual personal communication devices 22 also respectively include one or more cellular communications interfaces or Wi-Fi® communications interfaces as at least one of the communications interfaces 280. The supporter personal communication devices 20 or individual personal communication devices 22 can communicate the information to the recovery server 28 via a global system for mobile devices (“GSM”) by communications interface 280.

At least one of the supporter personal communication devices 20 or individual personal communication devices 22 also respectively include one or more user interfaces 286a-286n (collectively 286; only two shown), touch screens or keyboards 290a-290n (collectively 290; only two shown), or pointers (e.g., mice) 288a-288n (collectively 288; only two shown). A system operator such as a supporter 10 or individual 12 can enter commands and information into the respective supporter personal communication devices 20 or individual personal communication devices 22 by providing input to a user interface 286 through respective input devices, such as the touch screens or keyboards 290 and/or the pointers 288. Other optional input devices can include a microphone, joystick, game pad, tablet, scanner, etc. These and other input devices are communicably coupled to the processor 270 through an interface such as a universal serial bus (“USB”) interface that couples to the system bus 268, although other interfaces such as a parallel port, a game port or a wireless interface or serial port can be used. At least one of the supporter personal communication devices 20 or individual personal communication devices 22 also respectively include one or more displays 284a-284n (collectively 284; only two shown). The display 284 is coupled to the system bus 268. The display 284 can include a holographic display or another type of display (e.g., liquid crystal display). The supporter personal communication devices 20 or individual personal communication devices 22 can respectively include other optional output devices, such as speakers, printers, etc.

At least one of the supporter personal communication devices 20 or individual personal communication devices 22 also respectively include one or more geolocation systems 282a-282n (collectively 282; only two shown). At least one of the geolocation systems 282 can include a global positioning system (GPS). The GPS can couple to the system bus 268.

At least one of the supporter personal communication devices 20 or individual personal communication devices 22 also respectively include one or more sensors 292a-292n (collectively 292; only two shown). At least one of the sensors 292 can include at least one biosensor such as at least one accelerometer, heart rate supporter, blood content supporter, blood pressure supporter, or sleep tracker. At least one of the sensors 292 may be integral to the respective one of the individual personal communication devices 22 or supporter personal communication devices 20, and can communicatively couple to the at least one processor 272 via the system bus 268. Additionally or alternatively, at least one of the sensors 292 may include at least one wearable device (e.g., bracelet, anklet, watch, necklace) and can communicatively couple to the at least one processor 272 via one of the above-explained interfaces. For example, the respective one of the individual personal communication devices 22 or supporter personal communication devices 20 may be a wearable device that integrally contains at least one of the sensors 292. Additionally or alternatively, at least one of the respective one of the individual personal communication devices 22 or supporter personal communication devices 20 may include at least one implantable device that implants subcutaneously in a respective one of the individuals 12, and can integrally include at least one of the sensors 292.

Operation of the System

The term “addiction” at least means a primary, chronic disease of at least one of brain reward, motivation, memory, or related circuitry. Dysfunction in these circuits leads to characteristic biological, psychological, social, or spiritual manifestations. This is reflected in the individuals 8, 12 pathologically pursuing reward or relief by substance use or other behaviors. In particular, addiction is characterized by at least one of an inability to consistently abstain, impairment in behavioral control, craving, diminished recognition of significant problems with one's behaviors and interpersonal relationships, or a dysfunctional emotional response. Like other chronic diseases, addiction often involves cycles of relapse or remission. Without treatment or engagement in recovery activities, addiction is progressive or can result in disability or premature death.

The term “recovery” at least means a process of change through which a given one of the individuals 8, 12 achieves abstinence and improved health, wellness, and quality of life. Accordingly, the recovery server 28 is not any common modern server. Instead, the recovery server 28 is a server that is specifically programmed or structured to at least support a process of change through which the given one of the individuals 8, 12 achieves abstinence and improved health, wellness, and quality of life via, for example, at least one portion of at least one algorithm explained herein.

In particular, one or more of the at least one personal communication device of the individual or supporter or the at least one recovery server obtains personal data of the individual. For example, obtaining an individual's personal data can further include obtaining at least one sobriety birthday indicator. The at least one sobriety birthday indicator indicates at least one sobriety birthday of the individual. The at least one sobriety birthday is defined by at least one of i) at least one respective date that the individual most recently engaged or participated in at least one activity or behavior, or ii) at least one respective date that the individual first began a current duration of abstinence from the at least one activity or behavior. The at least one activity or behavior can be at least one to which the at least one individual has at least one addiction.

Obtaining an individual's personal data can further include obtaining at least one support routine indicator. The at least one support routine indicator indicates at least one course of treatment of addiction of the individual to at least one activity, behavior, substance, or item. The at least one course of treatment can include routine attendance of the at least one meeting. One or more of the at least one personal communication device of the individual or supporter or the at least one recovery server can obtain at least one portion of such data from at least one of the individual, a supporter, or a facility 6.

One or more of the at least one personal communication device of the individual or supporter or the at least one recovery server also obtains at least one of each indicator in a group of indicators that includes one or more selected from a meeting indicator, a clarity indicator, or a support contact indicator. The at least one meeting indicator indicates at least one meeting to be attended by the individual. The at least one meeting can include at least one support meeting, group session, or one-on-one session. The at least one clarity indicator indicates at least one clarity in which to at least one of attend, engage, or participate by the individual. The at least one clarity can include at least one spiritual clarity, mental clarity, or physical clarity. The at least one support contact indicator indicates at least one identifier or contact information of at least one supporter of the individual. One or more of the at least one personal communication device of the individual or supporter or the at least one recovery server can obtain at least one portion of such indicator(s) from at least one of the individual, a supporter, or a facility 6. For example, one or more of the at least one personal communication device of the individual or supporter or the at least one recovery server may obtain at least one portion of such indicators responsive to obtaining at least one location of the individual.

For example, one or more of the at least one personal communication device of the individual or supporter or the at least one recovery server can obtain, via at least one respective geolocation system that communicatively couples to the at least one personal communication device of the individual, at least one location indicator. The at least one location indicator indicates at least one estimated geographic location of the at least one personal communication device of the individual as estimated by the at least one respective geolocation system that communicatively couples to the at least one personal communication device of the individual.

One or more of the at least one personal communication device of the individual or supporter or the at least one recovery server also creates at least one individual profile of the individual based at least in part on at least one of the individual's personal data, the at least one meeting indicator, the at least one clarity indicator, or the at least one support contact indicator. For example, one or more of the at least one personal communication device of the individual or supporter or the at least one recovery server may create or adjust the profile based at least in part on the stage of addiction of the individual, the level of care that the individual requires, the type of addiction, the duration of the individual's participation in a rehabilitation program prior to implementing the system 2, the duration of the individual's engagement with the system 2, or the level of progress of the individual with regard to the system 2.

One or more of the at least one personal communication device of the individual or supporter or the at least one recovery server stores the at least one individual profile of the individual in one or more of the at least one respective nontransitory computer-readable memory of the at least one personal communication device or the at least one nontransitory computer-readable memory of the at least one recovery server.

One or more of the at least one personal communication device of the individual or supporter or the at least one recovery server can also determine at least one progress threshold based at least in part on the at least one individual profile of the individual. Additionally or alternatively, the progress threshold can be uniform for each of the individuals. One or more of the at least one personal communication device of the individual or supporter or the at least one recovery server obtains at least one of each respective indicator in an indicator group of indicators that includes one or more selected from meeting attendance indicator, clarity success indicator, or supporter communication indicator. The at least one meeting attendance indicator indicates whether the individual at least one of attends or attended one or more of the at least one meeting of the one or more of the at least one meeting. The at least one clarity success indicator indicates whether the individual at least one of attends, participates in, engages in, attended, participated in, or engaged in one or more of the at least one clarity. The at least one supporter communication indicator indicates whether the individual at least one of communicates or communicated with one or more of the at least one supporter of the individual.

One or more of the at least one personal communication device of the individual or supporter or the at least one recovery server also, in response to one or more of the at least one personal communication device of the individual or supporter or the at least one recovery server obtaining the at least one meeting attendance indicator, determines whether the individual at least one of attends or attended the one or more of the at least one meeting. One or more of the at least one personal communication device of the individual or supporter or the at least one recovery server, in response to one or more of the at least one personal communication device of the individual or supporter or the at least one recovery server obtaining the at least one clarity success indicator, determines whether the individual at least one of attends, participates in, engages in, attended, participated in, or engaged in the one or more of the at least one clarity of the one or more of the at least one clarity. One or more of the at least one personal communication device of the individual or supporter or the at least one recovery server, in response to one or more of the at least one personal communication device of the individual or supporter or the at least one recovery server obtaining the at least one supporter communication indicator, determines whether the individual at least one of communicates or communicated with the one or more of the at least one supporter of the individual.

For example, one or more of the at least one personal communication device of the individual or supporter or the at least one recovery server can also determine at least one scheduled respective date, respective time, or respective location of each of the at least one meeting. One or more of the at least one personal communication device of the individual or supporter or the at least one recovery server can determine whether the at least one estimated geographic location of the at least one personal communication device of the individual at least one of coincides or coincided with the at least one respective location of the one or more of the at least one meeting at the at least one respective date or respective time of the one or more of the at least one meeting. One or more of the at least one personal communication device of the individual or supporter or the at least one recovery server can also determine whether the at least one respective geolocation system that communicatively couples to the at least one personal communication device of the individual is enabled. One or more of the at least one personal communication device of the individual or supporter or the at least one recovery server can, in response to the at least one respective geolocation system that communicatively couples to the at least one personal communication device of the individual not being enabled, cause at least one display, via the respective display of the at least one personal communication device of the individual, of at least one geolocation enablement notification that at least one of instructs or prompts the individual to enable the at least one geolocation system that communicatively couples to the at least one personal communication device of the individual.

As another example, one or more of the at least one personal communication device of the individual or supporter or the at least one recovery server can also determine at least one respective clarity biometric value of each of the at least one clarity. The at least one respective clarity biometric value can indicate at least one estimated respective biometric of the individual while the individual at least one of attends, participates in, or engages in each of the at least one clarity. One or more of the at least one personal communication device of the individual or supporter or the at least one recovery server can obtain, via at least one respective biometric sensor that communicatively couples to the at least one personal communication device of the individual, at least one biometric indicator. The at least one biometric indicator indicates at least one estimated biometric of the individual as estimated by the at least one respective biometric sensor that communicatively couples to the at least one personal communication device of the individual. One or more of the at least one personal communication device of the individual or supporter or the at least one recovery server can determine whether the at least one estimated biometric of the individual coincides or coincided with the at least one estimated respective biometric of the individual while the individual attends, participates in, or engages in the one or more of the at least one clarity. One or more of the at least one personal communication device of the individual or supporter or the at least one recovery server can determine the at least one respective clarity biometric value of each of the at least one clarity based at least in part on at least one of i) at least one historical estimated respective biometric of the individual while the individual attends, participates in, or engages in each of the at least one clarity or, ii) at least one portion of the at least one individual profile of the individual. At least one spiritual clarity can include at least one of prayer, meditation, or religious service. At least one mental clarity can include at least one of creative exercise, reading, or writing. At least one physical clarity can include at least one of physical exercise, hiking, or sleeping.

As an additional example, one or more of the at least one personal communication device of the individual or supporter or the at least one recovery server can also analyze at least one telephone call, text message, or email that the at least one personal communication device of the individual at least one of transmits or receives. One or more of the at least one personal communication device of the individual or supporter or the at least one recovery server can determine whether the at least one analyzed telephone call, text message, or email includes the at least one supporter of the individual as at least one recipient or sender of the at least one analyzed telephone call, text message, or email.

Additionally or alternatively, one or more of the at least one personal communication device of the individual or supporter or the at least one recovery server can obtain, via at least one respective geolocation system that communicatively couples to the at least one personal communication device of the individual, at least one individual location indicator. The at least one individual location indicator can indicate at least one estimated individual geographic location of the at least one personal communication device of the individual as estimated by the at least one respective geolocation system that communicatively couples to the at least one personal communication device of the individual. One or more of the at least one personal communication device of the individual or supporter or the at least one recovery server can obtain, via at least one respective geolocation system that communicatively couples to the at least one personal communication device of the at least one supporter of the individual, at least one supporter location indicator. The at least one supporter location indicator indicates at least one estimated supporter geographic location of the at least one personal communication device of the at least one supporter of the individual as estimated by the at least one respective geolocation system that communicatively couples to the at least one personal communication device of the at least one supporter of the individual. One or more of the at least one personal communication device of the individual or supporter or the at least one recovery server can determine whether the at least one estimated individual geographic location of the at least one personal communication device of the individual at least one of coincides or coincided with the at least one estimated supporter geographic location of the at least one personal communication device of the at least one supporter of the individual.

Additionally or alternatively, one or more of the at least one personal communication device of the individual or supporter or the at least one recovery server can obtain, via at least one respective near field communication (NFC) system or personal area network (PAN) system that communicatively couples to the at least one personal communication device of the individual, at least one NFC or PAN communication indicator. The at least one NFC or PAN communication indicator indicates at least one identifier of at least one NFC system, PAN system, or personal communication device of another to which the NFC or PAN system that communicatively couples to the at least one personal communication device of the individual communicatively couples. One or more of the at least one personal communication device of the individual or supporter or the at least one recovery server can determine whether the at least one identifier of the at least one NFC system, PAN system, or personal communication device of the other matches at least one identifier of at least one NFC system, PAN system, or personal communication device of the at least one supporter of the individual.

One or more of the at least one personal communication device of the individual or supporter or the at least one recovery server can also generate at least one accountability score of the individual based at least in part on at least one of i) whether the individual at least one of attends or attended the one or more of the at least one meeting, ii) whether the individual at least one of attends, participates in, engages in, attended, participated in, or engaged in the one or more of the at least one clarity, or iii) whether the individual at least one of communicates or communicated with the one or more of the at least one supporter of the individual.

Importantly, one or more of the at least one personal communication device of the individual or supporter or the at least one recovery server can additionally or alternatively determine whether a given individual at least one of i) whether the individual at least one of attends or attended the one or more of the at least one meeting, ii) whether the individual at least one of attends, participates in, engages in, attended, participated in, or engaged in the one or more of the at least one clarity, or iii) whether the individual at least one of communicates or communicated with the one or more of the at least one supporter of the individual, based at least in part on any act explained above with regard to at least one other determination explained above. For example, one or more of the at least one personal communication device of the individual or supporter or the at least one recovery server can determine whether the individual at least one of attends or attended the one or more of the at least one meeting based at least in part on acts similar to those explained above with regard to the NFC or PAN system. As another example, one or more of the at least one personal communication device of the individual or supporter or the at least one recovery server can determine whether the individual at least one of attends, participates in, engages in, attended, participated in, or engaged in the one or more of the at least one clarity based at least in part on acts similar to those explained above with regard to the NFC or PAN system. Such NFC or PAN systems can alternatively be Wi-Fi® systems. Additionally or alternatively to making such determinations based at least in part on communicative coupling to another personal communication device, such determinations can be based at least in part on whether communicatively coupling occurs with regard to a stationary device at one or more of the facilities 6. As an additional example, one or more of the at least one personal communication device of the individual or supporter or the at least one recovery server can determine whether the individual at least one of attends, participates in, engages in, attended, participated in, or engaged in the one or more of the at least one clarity based at least in part on acts similar to those explained above with regard to the geolocation system.

One or more of the at least one personal communication device of the individual or supporter or the at least one recovery server also determines whether one or more of the accountability score of the individual at least one of equals or exceeds one or more of the at least one progress threshold.

One or more of the at least one personal communication device of the individual or supporter or the at least one recovery server also, in response to the one or more of the at least one accountability score of the individual at least one of equaling or exceeding the one or more of the at least one progress threshold, provides at least one progress success indicator to one or more of the at least one personal communication device of the individual or at one personal communication device of the at least one supporter of the individual. The at least one progress success indicator causes the one or more of the at least one personal communication device of the individual or the at least one personal communication device of the at least one supporter of the individual to display via the at least one respective display of the one or more of the at least one personal communication device of the individual or the at least one personal communication device of the at least one supporter of the individual at least one progress success notification that indicates to at least one of the individual or the at least one supporter of the individual that the one or more of the at least one accountability score of the individual at least one of equals or exceeds the one or more of the at least one progress threshold.

One or more of the at least one personal communication device of the individual or supporter or the at least one recovery server also, in response to the one or more of the at least one accountability score of the individual failing to at least one of equal or exceed the one or more of the at least one progress threshold, provides at least one progress support indicator to the one or more of the at least one personal communication device of the individual or supporter or the at least one personal communication device of the at least one supporter of the individual. The at least one progress support indicator causes the one or more of the at least one personal communication device of the individual or the at least one personal communication device of the at least one supporter of the individual to display via the at least one respective display of the one or more of the at least one personal communication device of the individual or the at least one personal communication device of the at least one supporter of the individual at least one progress support alert that alerts at least one of the individual or the at least one supporter of the individual that the one or more of the at least one accountability score of the individual fails to at least one of equal or exceed the one or more of the at least one progress threshold.

One or more of the at least one personal communication device of the individual or supporter or the at least one recovery server can also permit messaging between individual personal communication devices and supporter personal communication devices.

One or more of the at least one personal communication device of the individual or supporter or the at least one recovery server can also provide incentives to the individual responsive to the at least one accountability score of the individual at least one of equaling or exceeding the one or more of the at least one progress threshold. One or more of the at least one personal communication device of the individual or supporter or the at least one recovery server can provide incentives based at least in part on frequency or quantity of text messaging between the individual and at least one supporter or another individual. The incentives can include value or credit that permits the individual to obtain at least one item or experience in exchange for the value or credit.

One or more of the at least one personal communication device of the individual or supporter or the at least one recovery server can also obtain at least one shut-off alert. The shut-off alert indicates whether a respective individual personal communication device has been shut-off. Responsive to the shut-off alert, one or more of the at least one personal communication device of the individual or supporter or the at least one recovery server can provide at least one shut-off notification to at least another one or more of the at least one personal communication device of the individual or supporter or the at least one recovery server. The shut-off notification causes the other one or more of the at least one personal communication device of the individual or supporter or the at least one recovery server to display an indication that the individual may require support.

One or more of the at least one personal communication device of the individual or supporter or the at least one recovery server can also implement at least one of the above explained acts with regard to another individual additionally or alternatively to the supporter. For example, one or more of the at least one personal communication device of the individual or supporter or the at least one recovery server can determine whether the individual communicates with at least one other individual.

One or more of the at least one personal communication device of the individual or supporter or the at least one recovery server can also at least one of create or provide at least one of the above-explained alerts or notification on a regular basis or responsive to at least one particular trigger. One or more of the at least one personal communication device of the individual or supporter or the at least one recovery server can also at least one of create or provide at least one additional alert or notification on a regular basis or responsive to at least one particular trigger. One or more of the at least one personal communication device of the individual or supporter or the at least one recovery server can determine the frequency (e.g., every given number of hours, days, weeks, months) of the regular basis based at least in part on at least one stage of addiction of the individual, at least one level of care that the individual requires, or at least one level of accountability of the individual. One or more of the at least one personal communication device of the individual or supporter or the at least one recovery server can determine the trigger based at least in part on at least one stage of addiction of the individual, at least one level of care that the individual requires, or at least one level of accountability of the individual. One or more of the at least one personal communication device of the individual or supporter or the at least one recovery server can determine the accountability of the individual based at least in part on at least one of number or percentage of activities that the individual successfully achieved as scheduled as explained above. Additionally or alternatively, one or more of the at least one personal communication device of the individual or supporter or the at least one recovery server can determine the accountability of the individual based at least in part on such number or percentage over time (e.g., in a given hour, day, week, month). One or more of the at least one personal communication device of the individual or supporter or the at least one recovery server can also periodically or continuously adjust the frequency or trigger to evolve with the individual or recovery of the individual.

For example, one or more of the at least one personal communication device of the individual or supporter or the at least one recovery server can create or provide at least one alert or notification every four hours for a stage four alcoholic. One or more of the at least one personal communication device of the individual or supporter or the at least one recovery server can trigger creation or provision of at least one alert or notification responsive to a stage four alcoholic missing a single scheduled activity. Additionally or alternatively, one or more of the at least one personal communication device of the individual or supporter or the at least one recovery server can create or provide at least one alert or notification responsive to a given individual failing to achieve 60%-70% of activities scheduled for the given individual in a 48 hour period.

One or more of the at least one personal communication device of the individual or supporter or the at least one recovery server can also periodically or continuously adjust the schedule of the individual based at least in part on at least one change in accountability of the individual as explained above. Additionally or alternatively, one or more of the at least one personal communication device of the individual or supporter or the at least one recovery server can instantaneously adjust the schedule of the individual based at least in part on at least one estimated respective biometric of the individual. For example, responsive to the individual waking up with a high heart rate, one or more of the at least one personal communication device of the individual or supporter or the at least one recovery server can schedule more clarity time for the individual. As another example, one or more of the at least one personal communication device of the individual or supporter or the at least one recovery server may implement at least one virtual therapist (e.g., remotely located therapist or automated therapist that communicates with the individual via a respective display of the one or more of the at least one personal communication device of the individual or supporter or the at least one recovery server) responsive to at least one estimated respective biometric of the individual indicating at least one high stress level. Additionally or alternatively, one or more of the at least one personal communication device of the individual or supporter or the at least one recovery server can create or provide at least one alert or notification based at least in part on at least one obtained estimated respective biometric of the individual indicating a bad mood accompanied by a lack of desire by the individual to socialize (e.g., via obtained indicators that pertain to activity of the individual or via input by the individual).

One or more of the at least one personal communication device of the individual or supporter or the at least one recovery server can also include at least one geographical indicator in any of the alerts or notifications explained herein to thereby cause another one or more of the at least one personal communication device of the individual or supporter or the at least one recovery server to display a present location of the individual, thereby permitting another individual or a supporter to find the individual.

One or more of the at least one personal communication device of the individual or supporter or the at least one recovery server can also implement machine learning (e.g., artificial neural network) to evolve at least one portion of at least one algorithm explained herein over time with the individual. Additionally or alternatively, one or more of the at least one personal communication device of the individual or supporter or the at least one recovery server can implement at least one portion of at least one algorithm explained herein as at least one predictive algorithm that causes one or more of the at least one personal communication device of the individual or supporter or the at least one recovery server to preemptively implement at least one portion of at least one act explained herein. Such not only preempts difficult situations of the individual, but also permits one or more of the at least one personal communication device of the individual or supporter or the at least one recovery server to allocate resources to such at least one portion of the at least one act when such resources have relatively historically high levels of availability, thereby improving performance and minimum requirements of at least one of the supporter personal communication devices 20, individual personal communication devices 22, recovery server 28, or communications network 24.

One or more of the at least one personal communication device of the individual or supporter or the at least one recovery server causes any display explained herein via at least one aural, haptic, or visual display.

One or more of the at least one personal communication device of the individual or supporter or the at least one recovery server can obtain, with regard to any act explained herein, responsive to a push by at least another one or more of the at least one personal communication device of the individual or supporter or the at least one recovery server. Additionally or alternatively, one or more of the at least one personal communication device of the individual or supporter or the at least one recovery server can obtain, with regard to any act explained herein, by pulling from at least another one or more of the at least one personal communication device of the individual or supporter or the at least one recovery server. One or more of the at least one personal communication device of the individual or supporter or the at least one recovery server can provide, with regard to any act explained herein, responsive to a pull by at least another one or more of the at least one personal communication device of the individual or supporter or the at least one recovery server. Additionally or alternatively, one or more of the at least one personal communication device of the individual or supporter or the at least one recovery server can provide, with regard to any act explained herein, by pushing to at least another one or more of the at least one personal communication device of the individual or supporter or the at least one recovery server. Accordingly, at least by accepting at least one push, one or more of the at least one personal communication device of the individual or supporter or the at least one recovery server does not need to repeatedly request such, thereby reducing processing resource consumption (e.g., power, processing, bandwidth) and communication resource consumption (e.g., data usage, bandwidth). Also accordingly, at least by pulling, one or more of the at least one personal communication device of the individual or supporter or the at least one recovery server reduces a likelihood of receiving such while overloaded with other tasks that may prevent one or more of the at least one personal communication device of the individual or supporter or the at least one recovery server from accepting such.

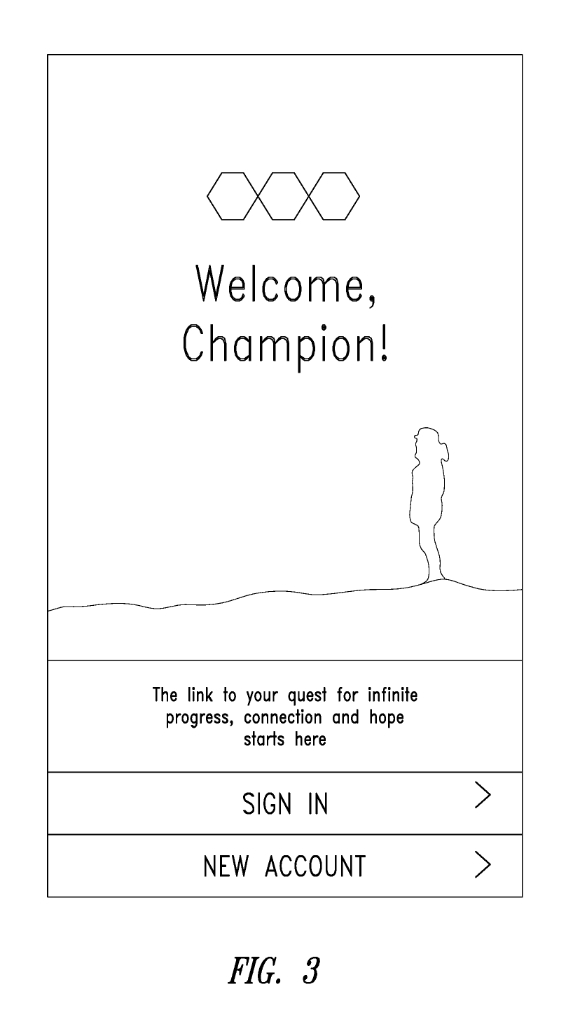

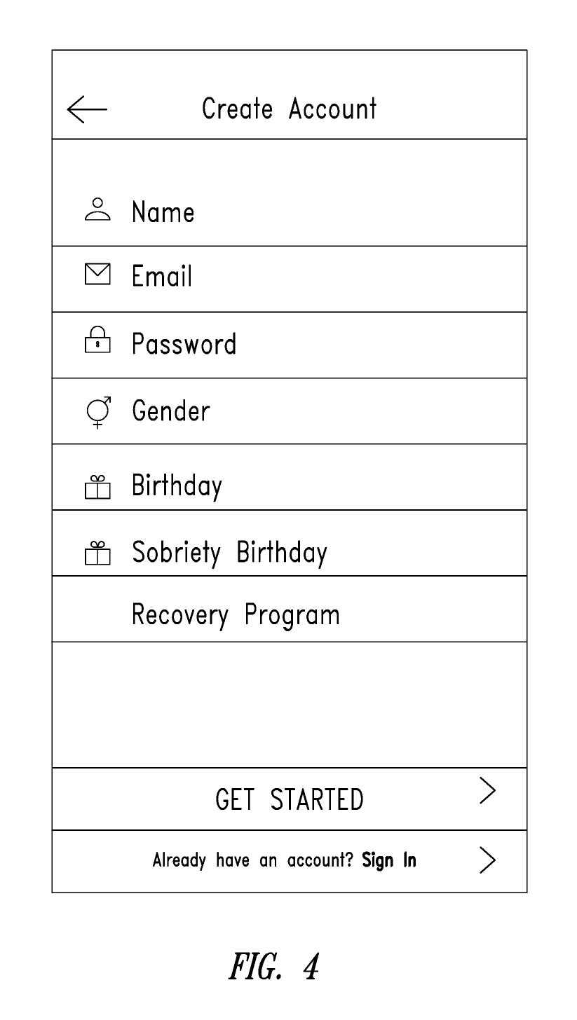

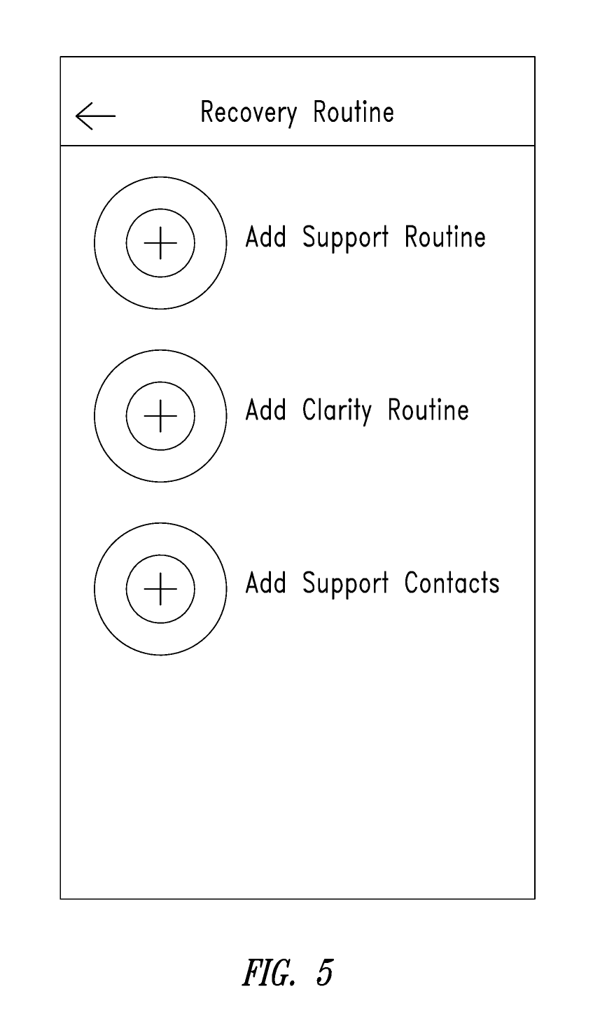

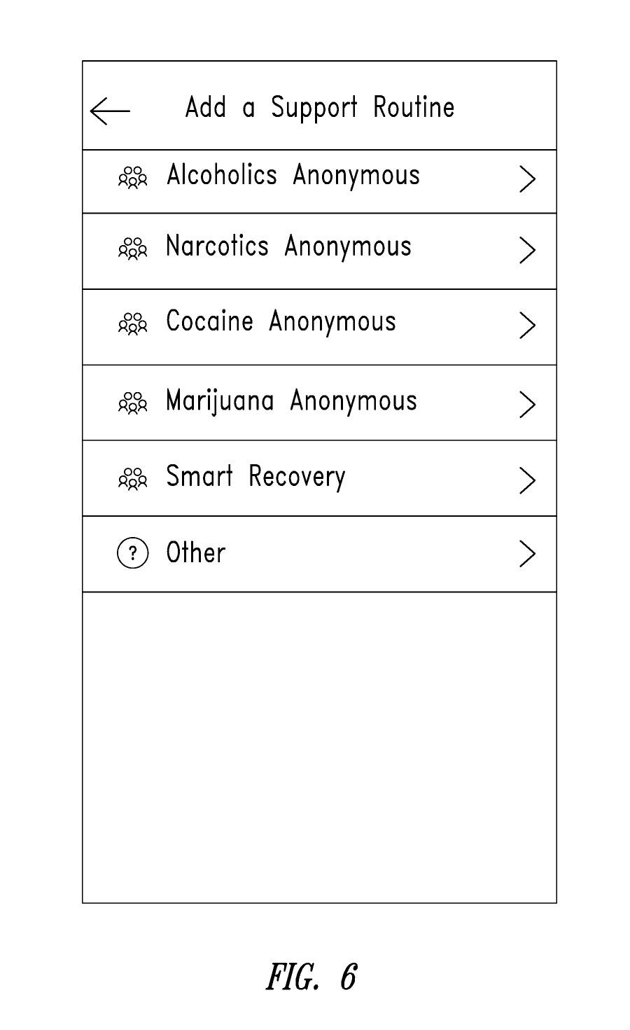

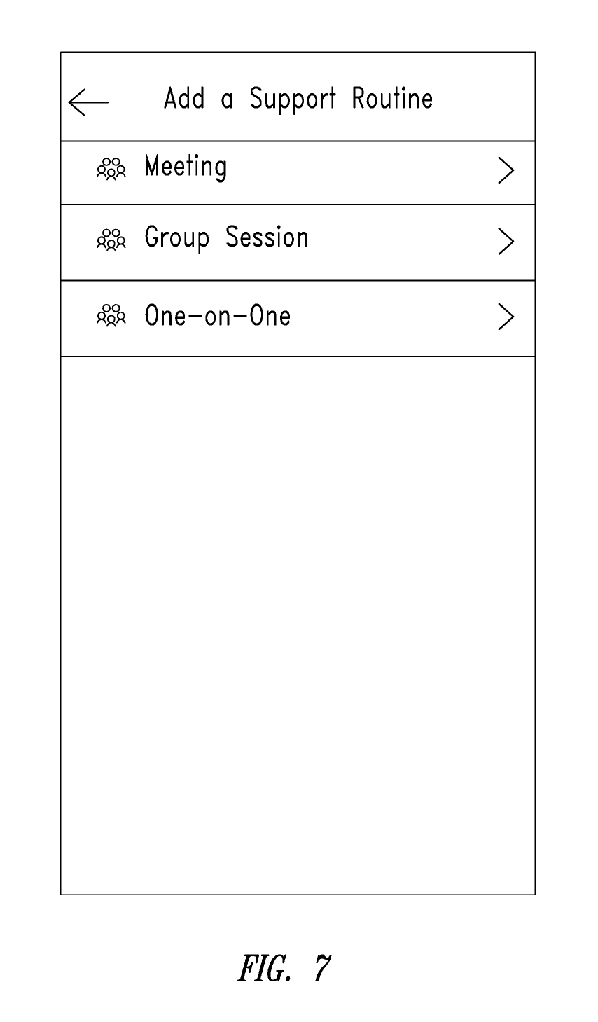

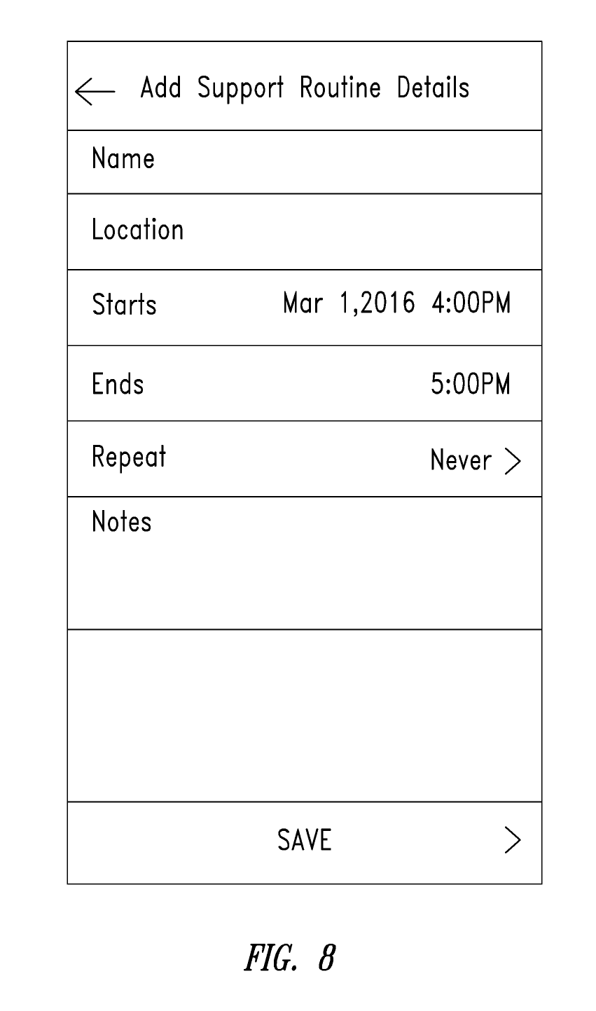

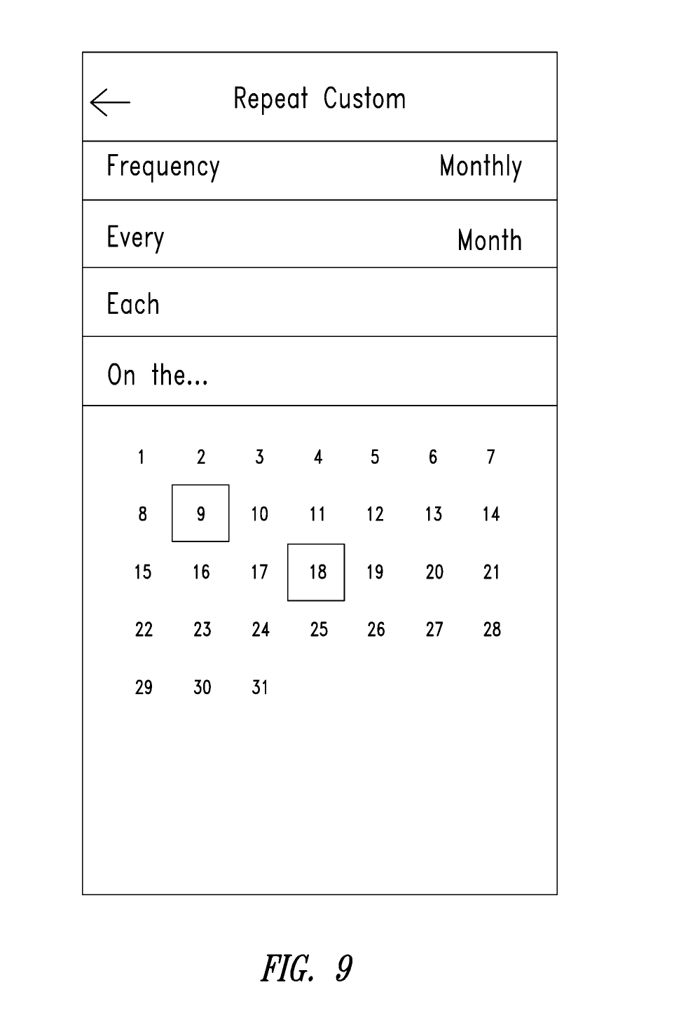

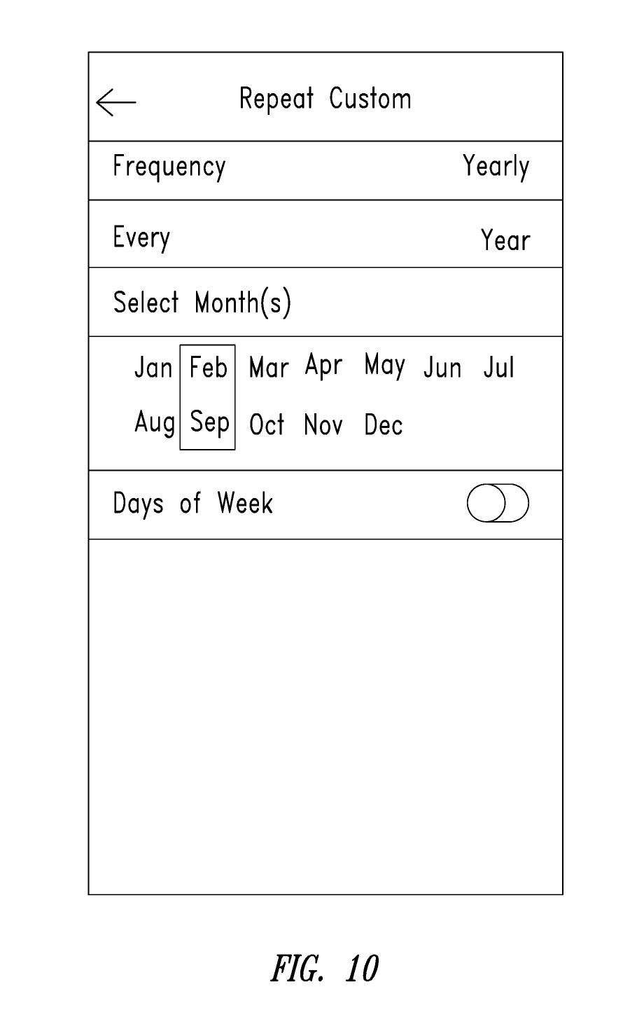

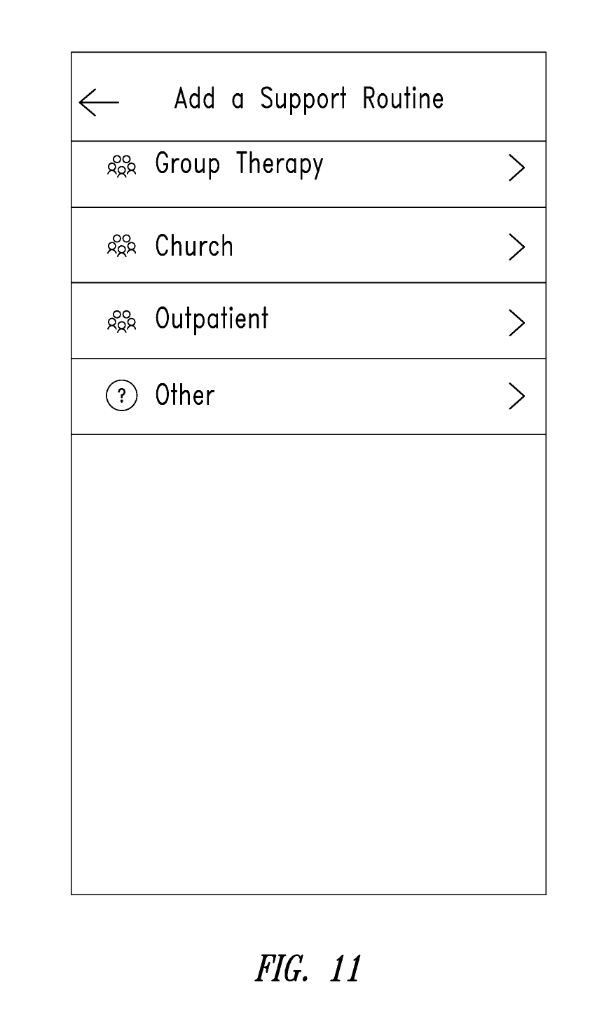

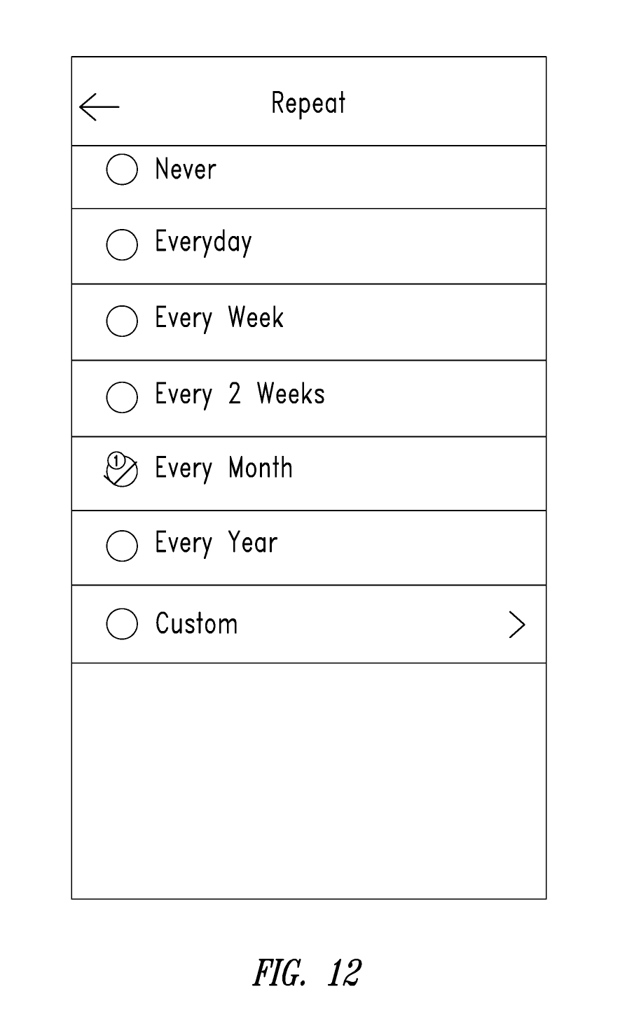

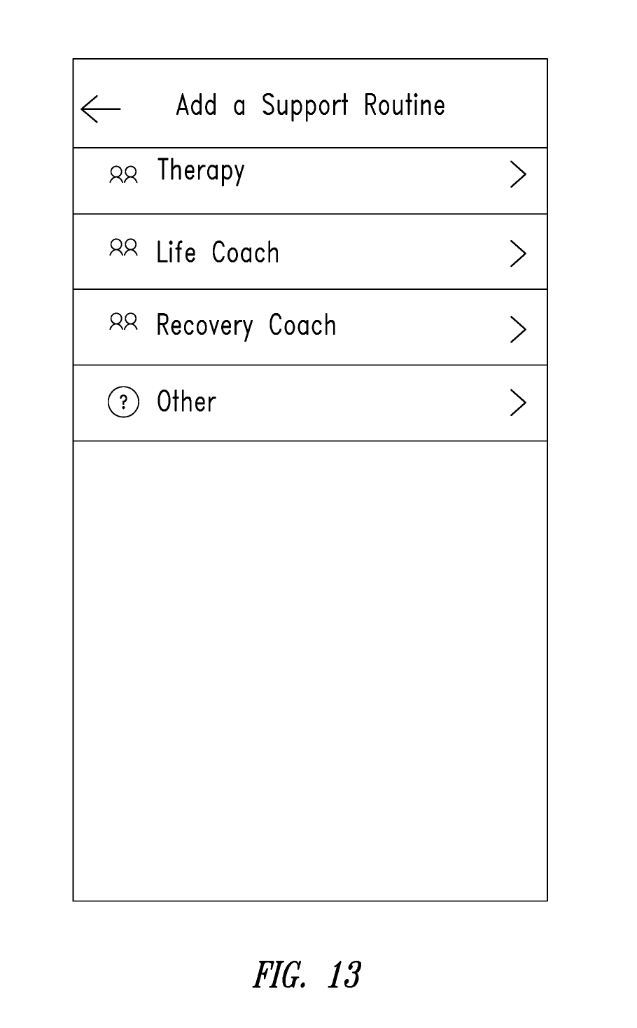

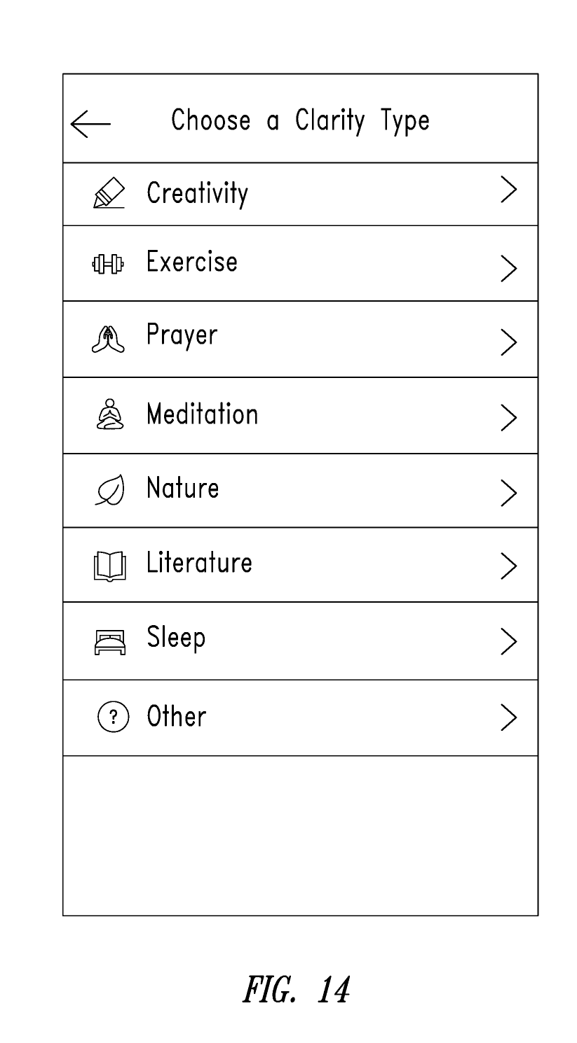

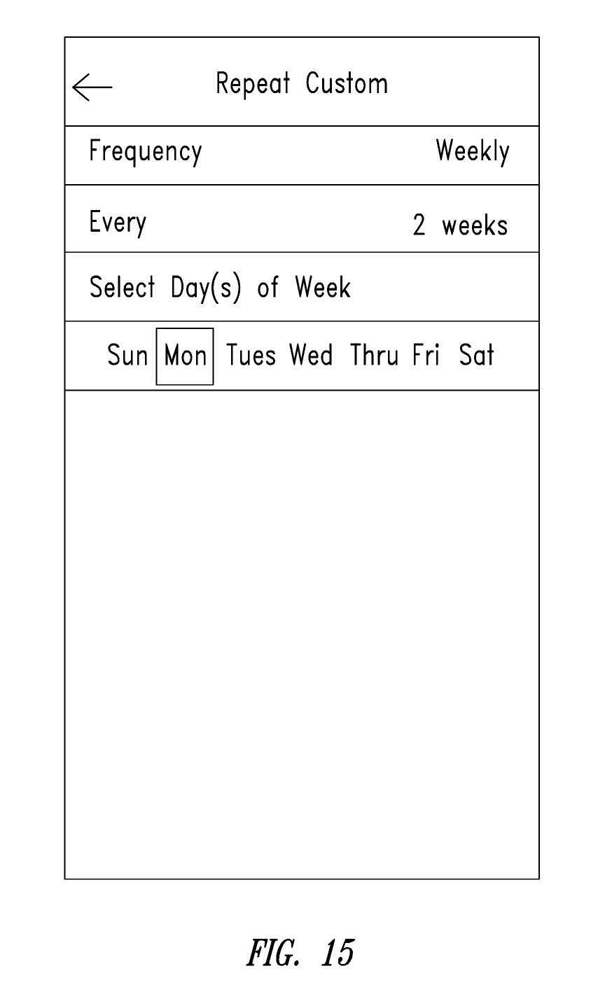

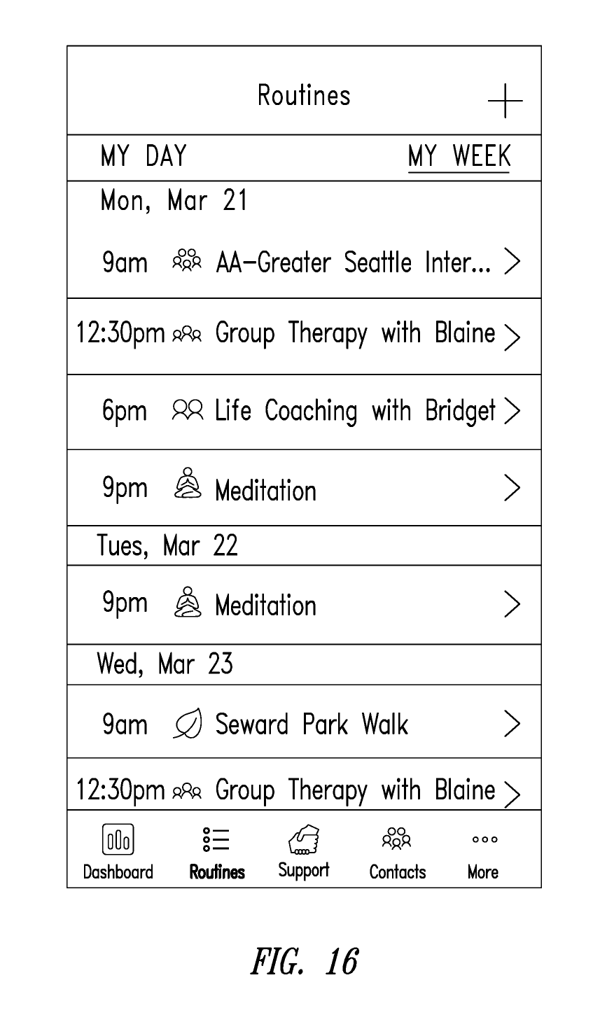

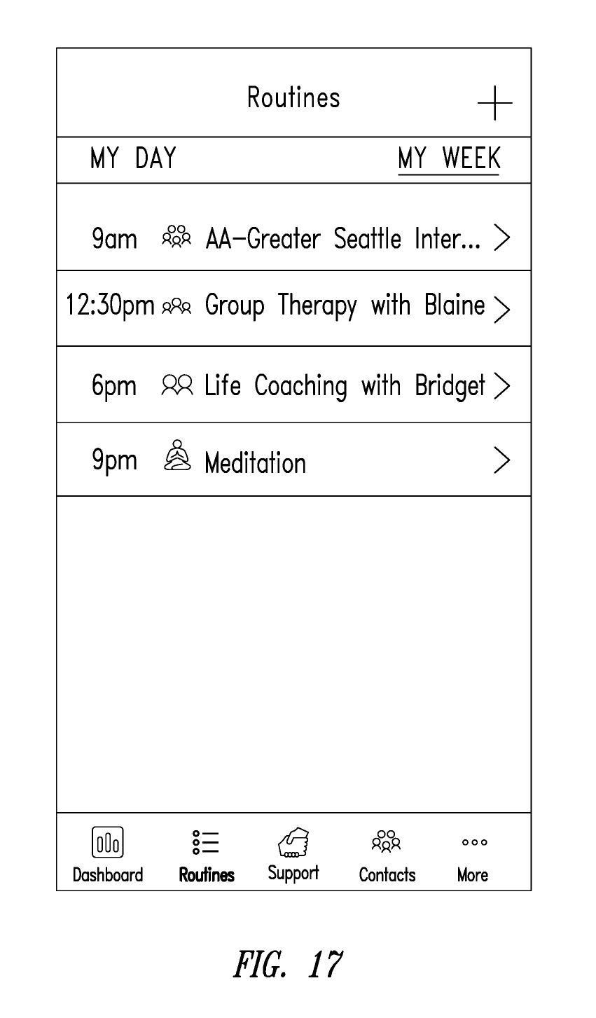

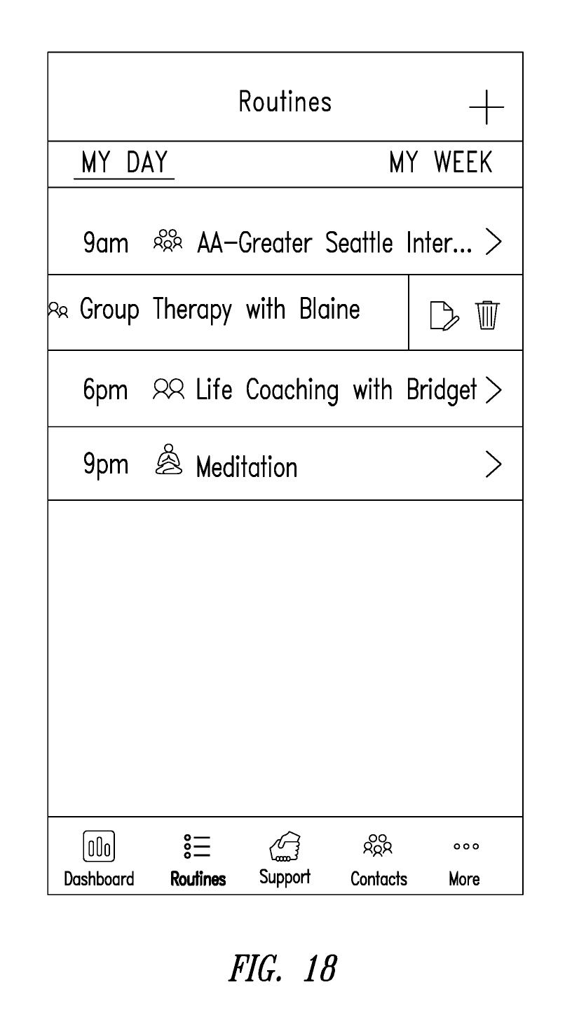

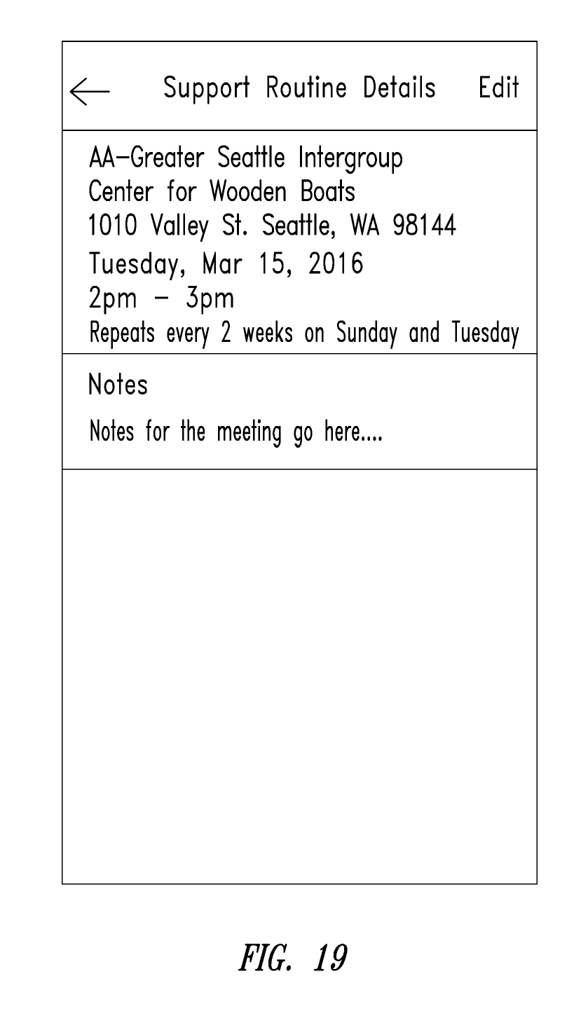

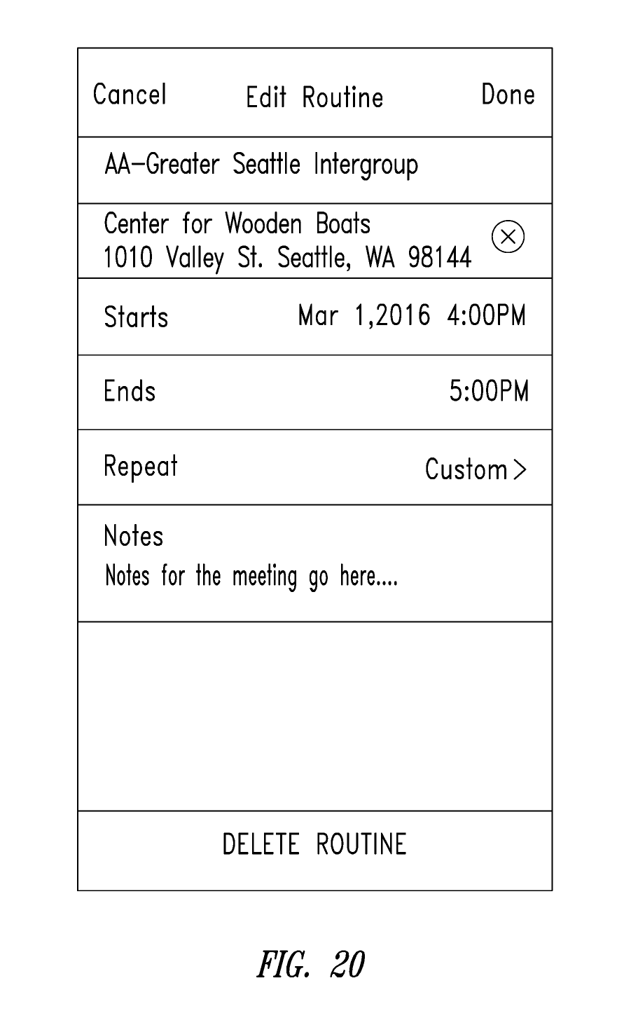

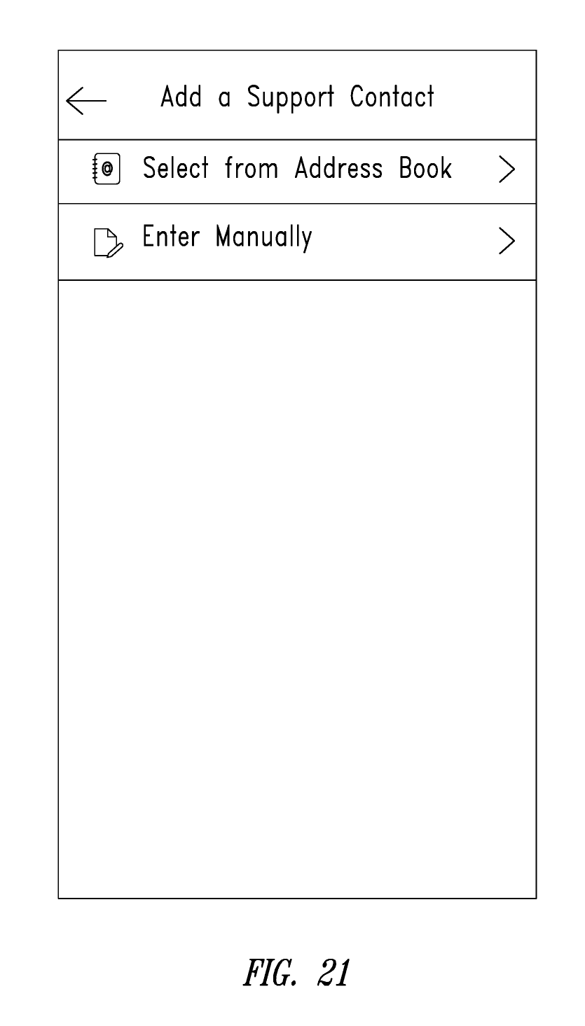

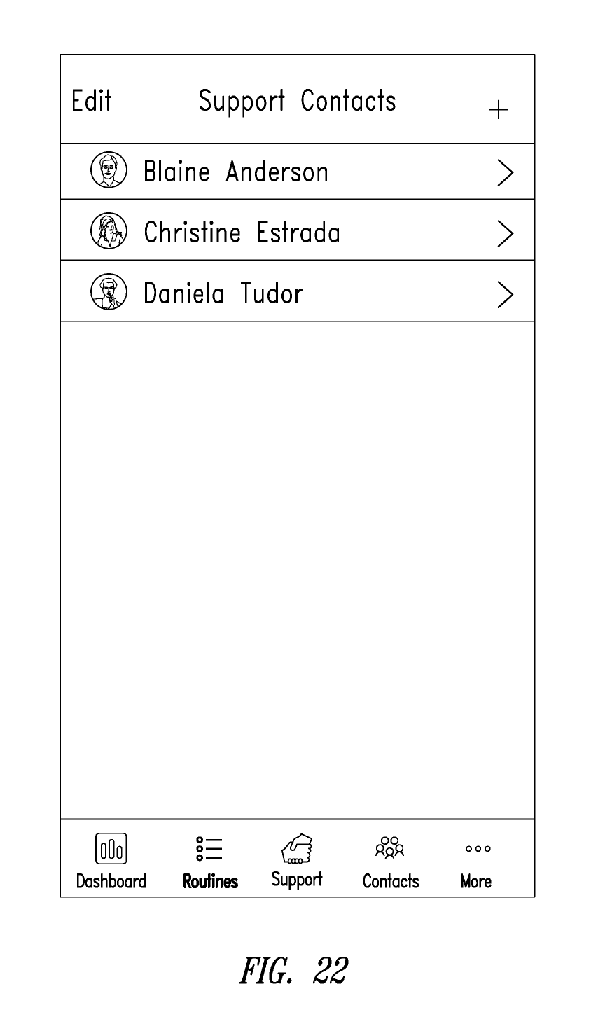

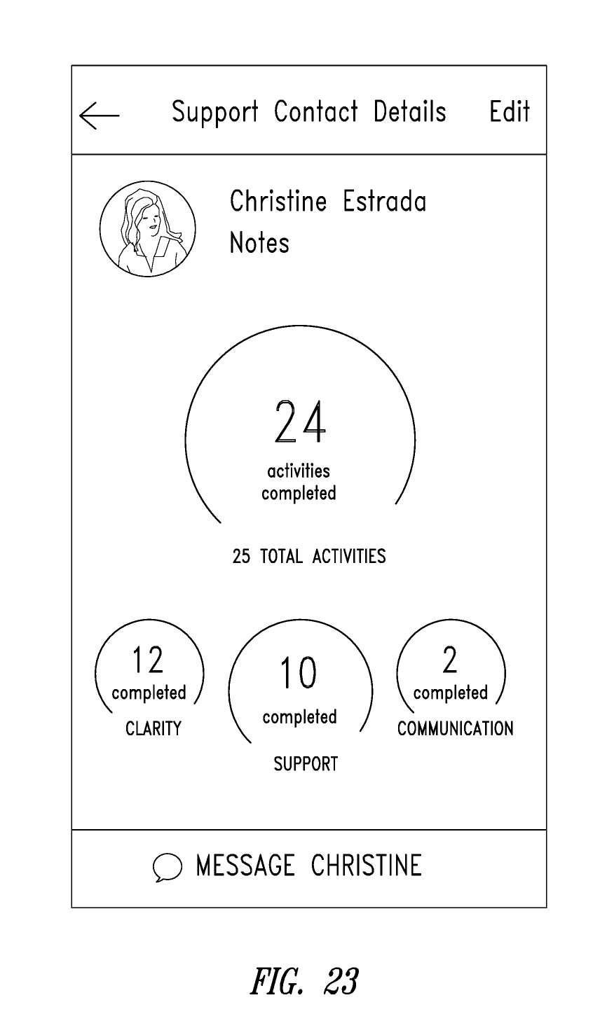

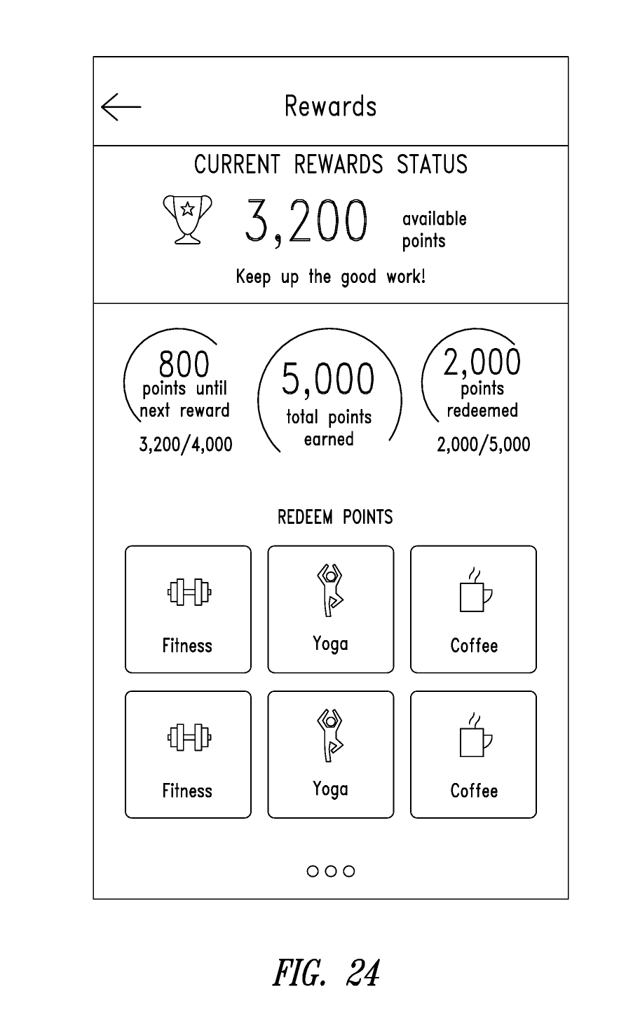

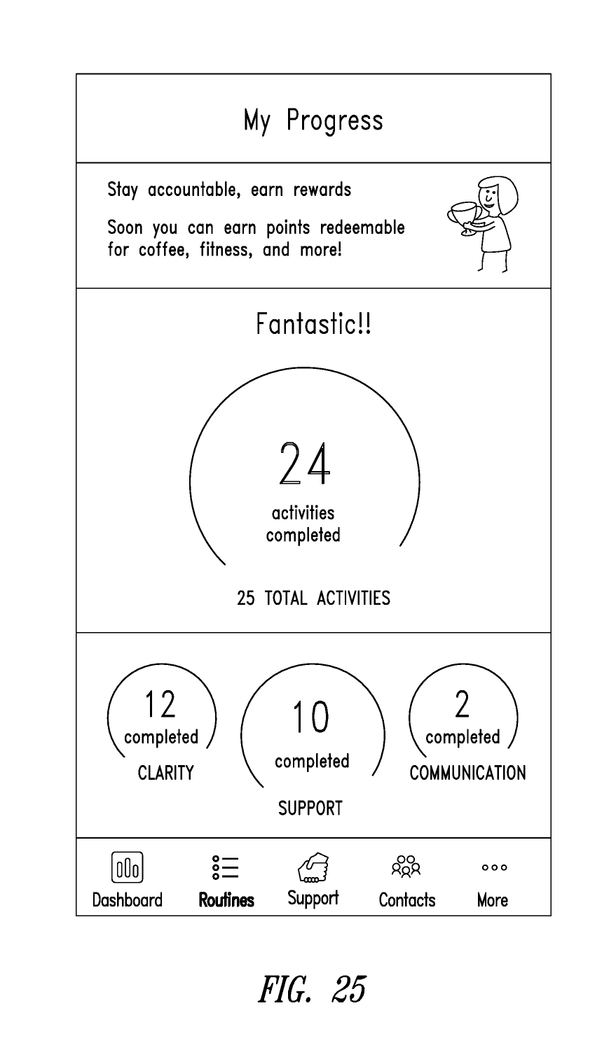

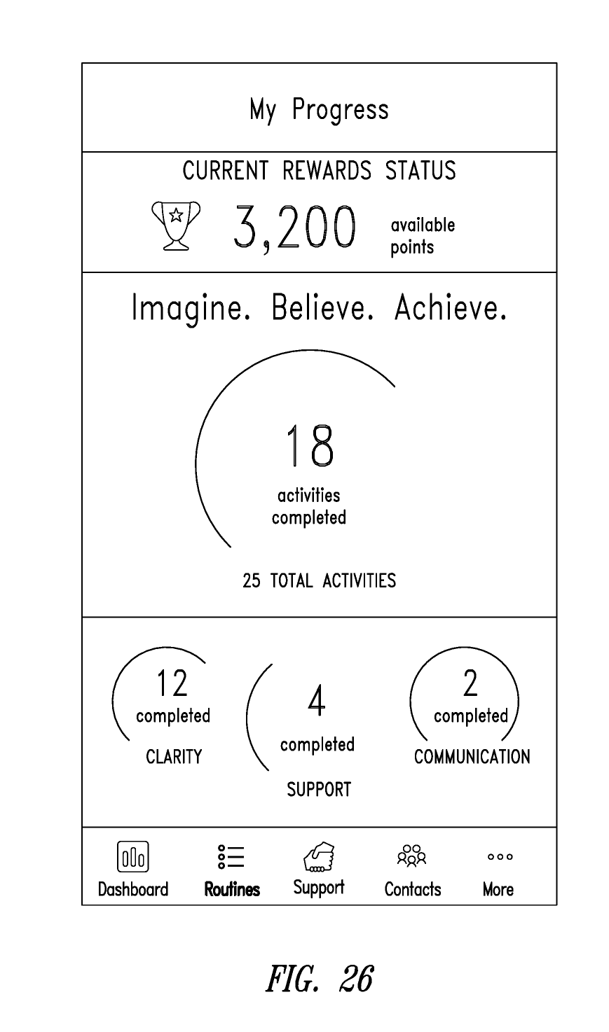

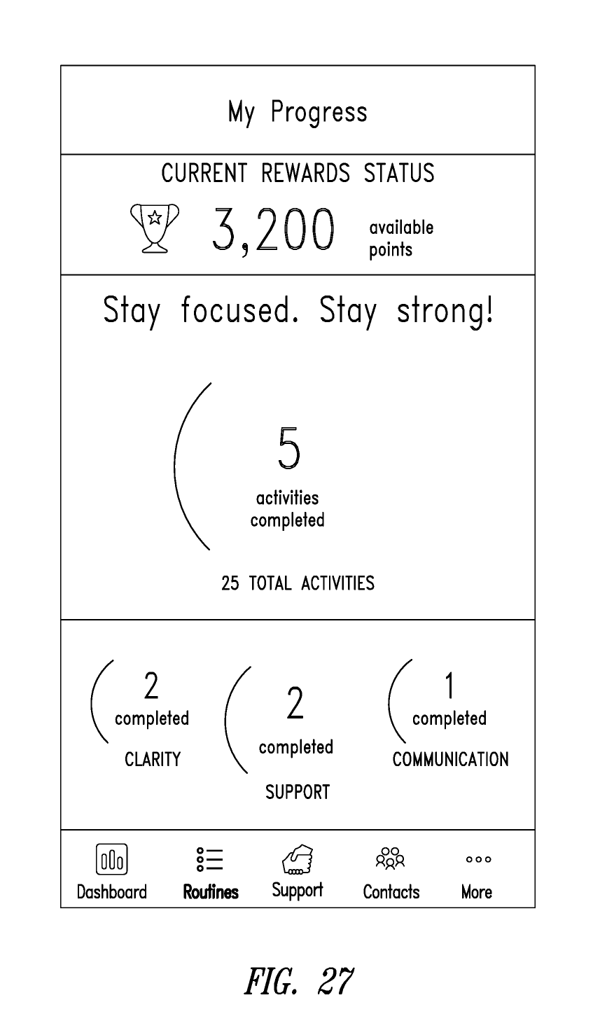

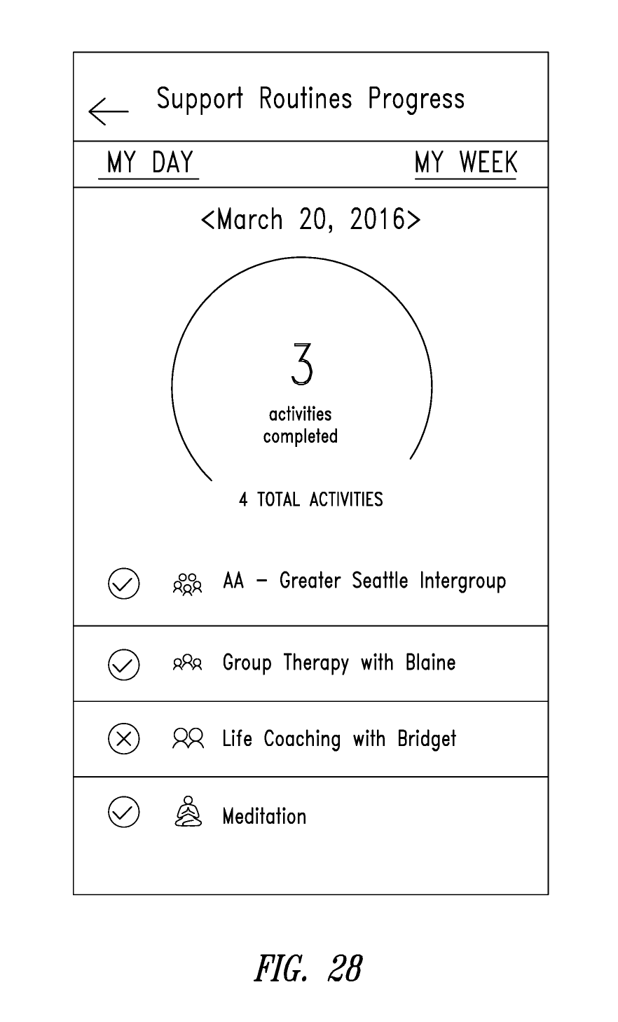

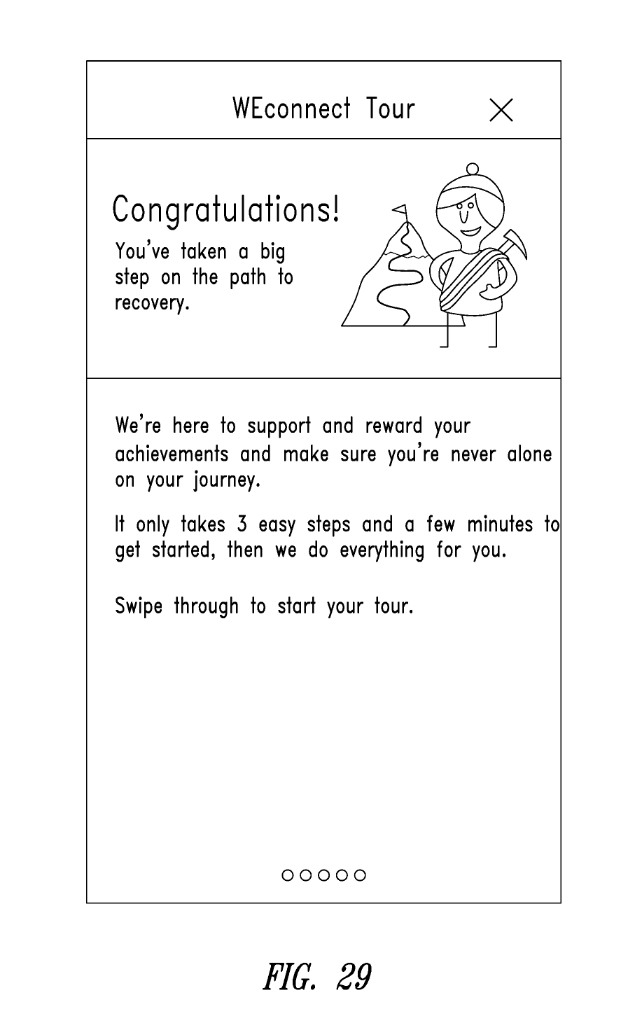

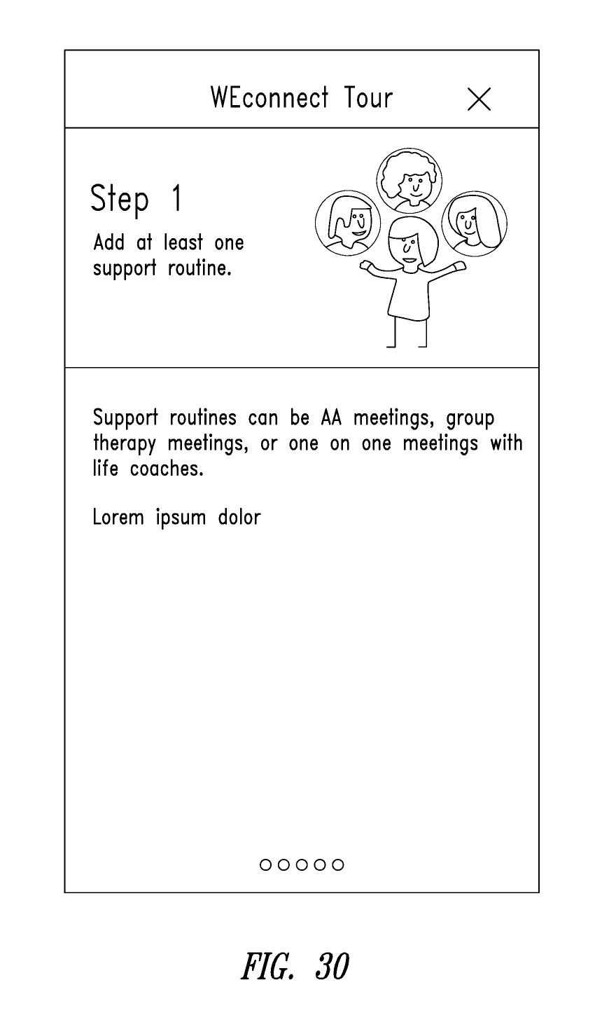

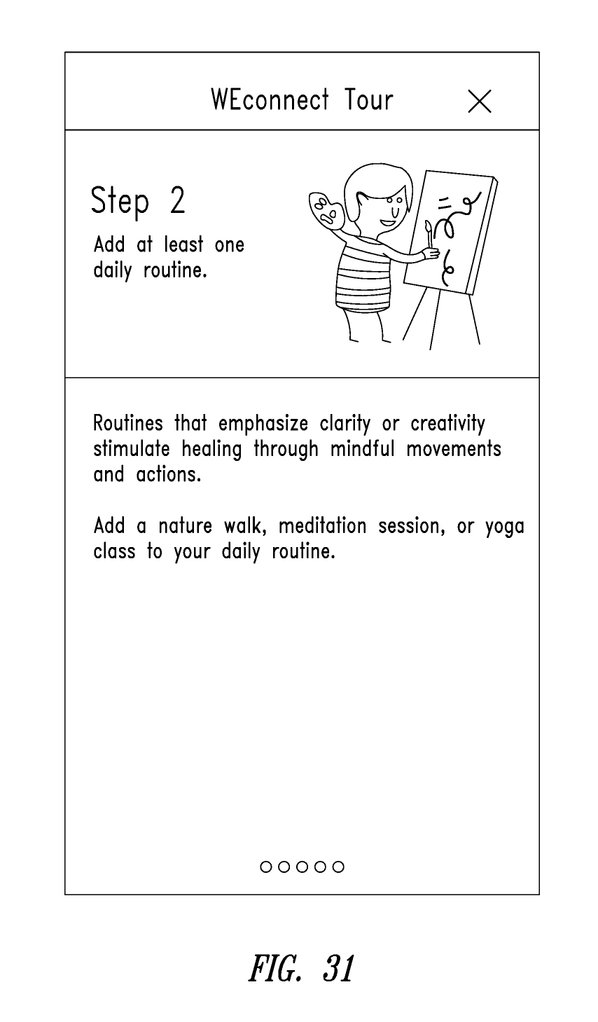

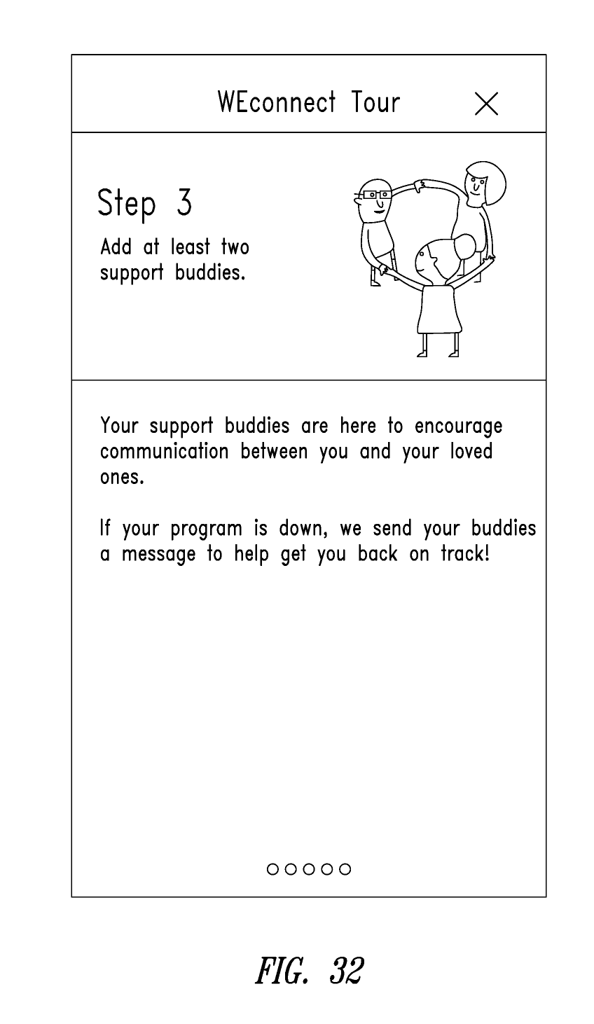

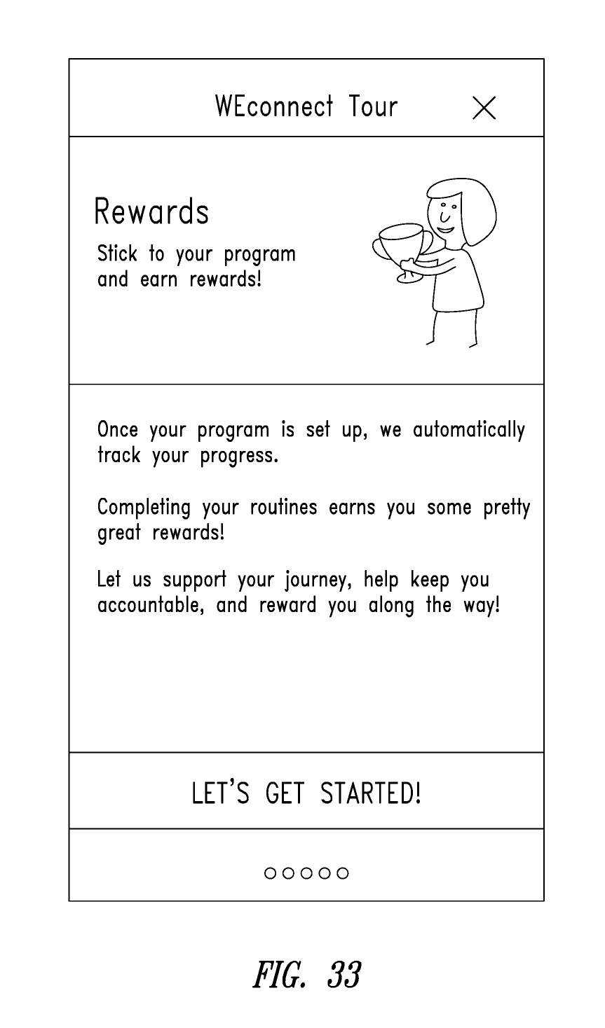

The recovery server 28 can include or communicably couple with at least one application server or Web server. The application or Web server provides recovery content (e.g., at least one recovery scheduler) to the given one of the individual personal communication devices 22 via the communications network 24. The recovery content activates at least one element (e.g., a browser that transforms the recovery content to interactive recovery content such as an interactive recovery scheduler responsive to the specific programming of the recovery content) of the given one of the individual personal communication devices 22 to cause at least one individual graphical interface (individual GUI) to display on the given one of the individual personal communication devices 22. The individual graphical interface can permit entry of at least one portion of the instructions via at least one interface of the given one of the individual personal communication devices 22. For example, the individual graphical interface can include at least one selectable or editable text box, check box, radio button, drop down list, etc. The individual graphical interface can permit submission of the recovery instructions. For example, the recovery content can have specific programming that causes the element to transform data input via the individual graphical interface to recovery instructions that have at least one of the illustrated or herein-explained formats. Such can involve comparing at least one piece of input data to at least one reference in at least one memory to populate or change at least one portion of the input data. Accordingly, at least because the recovery server 28 provides the recovery content to the given one of the individual personal communication devices 22, the recovery server 28 permits the given one of the individual personal communication devices 22 to have an increase in freedom with regard to storage allocation. For example, FIGS. 3-33 show respective screen shots of example instances of the individual graphical interface that the element of the given one of the individual personal communication devices 22 causes to display on the given one of the individual personal communication devices 22.

Additionally or alternatively, the given one of the individual personal communication devices 22 can store at least one individual application. The recovery server 28 can deploy at least one update to the individual application via the communications network 24. For example, the recovery server 28 can cause the individual application to transform the above-explained recovery content to interactive recovery content such as an interactive recovery scheduler. Responsive to executing the individual application, the given one of the individual personal communication devices 22 can display the above-explained individual graphical interface that permits entry of at least one portion of the instructions via at least one interface of the given one of the individual personal communication devices 22. The individual graphical interface can permit at least one submission of the recovery instructions as explained above. The given one of the individual personal communication devices 22 can store the individual application in local nontransitory computer-readable memory, thereby permitting operation of the recovery scheduler when the given one of the individual personal communication devices 22 communicably couples to the recovery server 28 and when the given one of the individual personal communication devices 22 fails to communicably couple to the recovery server 28 (e.g., dead zone, airplane mode, peak rates). The individual application causes the given one of the individual personal communication devices 22 to store the recovery instructions as at least one recovery instructions object. Responsive to detecting a lack of connection to the communications network 24, the individual application causes the given one of the individual personal communication devices 22 to store the recovery instructions object to local nontransitory computer-readable memory. The individual application causes the given one of the individual device or the individual personal communication devices 22 to communicably couple to the recovery server 28 responsive to detecting connection to the communications network 24. Responsive to such communicable coupling, the individual application causes the given one of the individual personal communication devices 22 to submit at least one of the recovery instructions or recovery instructions object to the recovery server 28 via the communications network 24. At least because the recovery server 28 enables the given one of the individual personal communication devices 22 to implement the recovery scheduler via the individual application, the recovery server 28 permits conservation of communication resources (e.g., data usage, bandwidth) until the given one of the individual personal communication devices 22 has an ability to communicably couple to the recovery server 28 via a faster or higher bandwidth connection (e.g., IEEE 802.11x data speed or bandwidth as compared to those of cellular data communication). For example, the individual application can cause the given one of the individual personal communication devices 22 to display at least one example instance of the individual graphical interface that the respective screen shots of FIGS. 3-33 show.

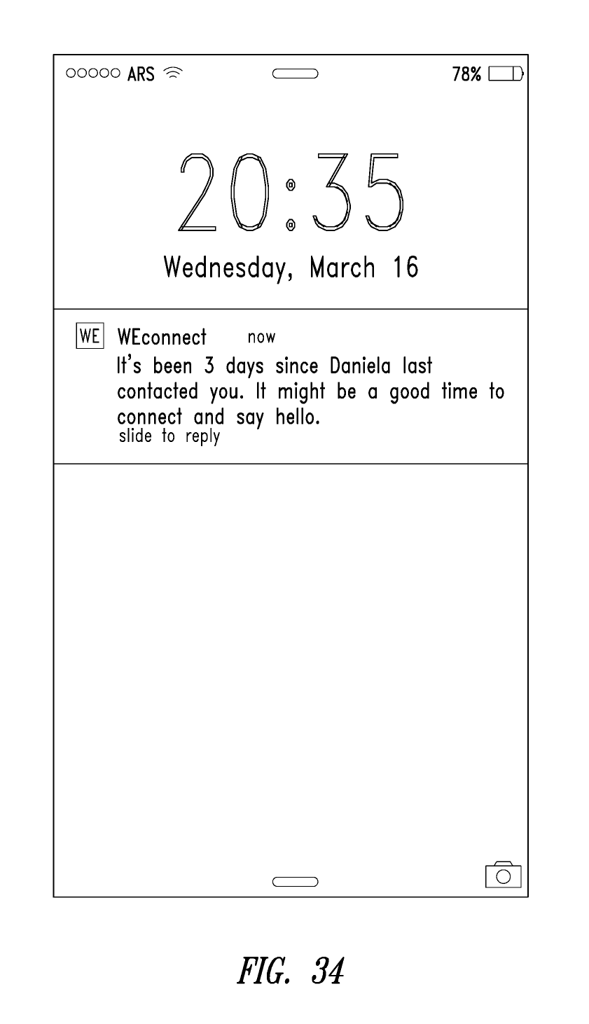

The recovery server 28 can include or communicably couple with the application server or Web server as explained above. The application or Web server provides supporter content (e.g., such can include at least one indicator pertaining to recovery of a logically associated individual 12) to the given one of the supporter personal communication devices 20 via the communications network 24. The supporter content activates at least one element (e.g., a browser that transforms the supporter content to interactive supporter content such as an interactive supporter interface responsive to the specific programming of the supporter content) of the given one of the supporter personal communication devices 20 to cause at least one supporter graphical interface (supporter GUI) to display on the given one of the supporter personal communication devices 20. Accordingly, at least because the recovery server 28 provides the supporter content to the given one of the supporter personal communication devices 20, the recovery server 28 permits the given one of the supporter personal communication devices 20 to have an increase in freedom with regard to storage allocation. For example, FIG. 34 shows a screen shot of an example instance of the supporter graphical interface that the element of the given one of the supporter personal communication devices 20 causes to display on the given one of the supporter personal communication devices 20.

Additionally or alternatively, the given one of the supporter personal communication devices 20 can store at least one supporter application. The recovery server 28 can deploy at least one update to the supporter application via the communications network 24. For example, the recovery server 28 can cause the supporter application to transform the above-explained supporter content to interactive supporter content such as an interactive supporter interface. Responsive to executing the supporter application, the given one of the supporter personal communication devices 20 can display the above-explained supporter graphical interface. The recovery server 28 can provide at least one piece of the recovery instructions to the given one of the supporter personal communication devices 20 via the communications network 24. The at least one piece of the recovery instructions can cause the given one of the supporter personal communication devices 20 to populate the supporter graphical interface. The supporter application can cause the given one of the supporter personal communication devices 20 to populate the supporter graphical interface with supporter content or interactive supporter content. At least because the recovery server 28 enables the given one of the supporter personal communication devices 20 to use the supporter interface via the supporter application, the recovery server 28 permits communicating the at least one indicator pertaining to recovery of a logically associated individual 12 to the given one of the supporter personal communication devices 20 without data indicative of graphical displays, thereby conserving communication resources. For example, the supporter application can cause the given one of the supporter personal communication devices 20 to display the example instance of the supporter graphical interface shown in the screen shot of FIG. 34.

In some implementations, at least one portion of at least one of the above-explained algorithms can operate continuously, or can operate as a plurality of threads of a multi-threaded process operating on suitable processor(s) (e.g., multi-core processor(s)).

The above description of illustrated implementations, including what is described in the Abstract, is not intended to be exhaustive or to limit the implementations to the precise forms disclosed. Although specific implementations of and examples are described herein for illustrative purposes, various equivalent modifications can be made without departing from the spirit and scope of the disclosure, as will be recognized by those skilled in the relevant art. The teachings provided herein of the various implementations can be applied to other systems, not necessarily the exemplary systems generally described above.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one implementation, the present subject matter can be implemented via application-specific integrated circuits (ASICs). However, those skilled in the art will recognize that the implementations disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

When logic is implemented as software and stored in memory, logic or information can be stored on any processor-readable medium for use by or in connection with any processor-related system or method. In the context of this disclosure, a memory is a processor-readable medium that is an electronic, magnetic, optical, or other physical device or means that contains or stores a computer and/or processor program. Logic and/or the information can be embodied in any processor-readable medium for use by or in connection with an instruction execution system, apparatus, or device, such as a computer-based system, processor-containing system, or other system that can fetch the instructions from the instruction execution system, apparatus, or device and execute the instructions associated with logic and/or information.

While various exemplary methods and algorithms are described above, it is noted that in each method or algorithm certain acts can be omitted, additional acts may be added, or both. Those of skill in the art will also appreciate that the illustrated order of the acts is shown for exemplary purposes, and the order of acts can change in alternative implementations.

In the context of this specification, a “non-transitory processor-readable medium” can be any element that can store the program associated with logic and/or information for use by or in connection with the instruction execution system, apparatus, and/or device. The processor-readable medium can be, for example, but is not limited to, an electronic, magnetic, optical, electromagnetic, infrared, or semiconductor system, apparatus or device. More specific examples (a non-exhaustive list) of the computer readable medium would include the following: a portable computer diskette (magnetic, compact flash card, secure digital, or the like), a random access memory (RAM), a read-only memory (ROM), an erasable programmable read-only memory (EPROM, EEPROM, or Flash memory), a portable compact disc read-only memory (CDROM), digital tape, and other non-transitory media.

While examples are explained in terms of one or more databases, in some implementations other collections of data can be employed, such collections of data generally being denominated as data stores.

In addition, those skilled in the art will appreciate that the mechanisms taught herein are capable of being distributed as a program product in a variety of forms, and that an illustrative implementation applies equally regardless of the particular type of non-transitory processor-readable media used to actually carry out the distribution.

The various implementations described above can be combined to provide further implementations. Aspects of the implementations can be modified, if necessary, to employ systems, circuits, and concepts to provide yet further implementations.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Application Ser. No. 62/336,113 filed May 13, 2016, are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
receiving, by at least one recovery server, personal data of an individual, the individual having at least one addiction to at least one activity, behavior, item, or substance;
receiving, by the at least one recovery server, at least one meeting indicator, the at least one meeting indicator indicative of at least one meeting to attend by the individual, the at least one meeting including at least one support meeting, group session, or one-on-one session;
receiving, by the at least one recovery server, at least one clarity indicator, the at least one clarity indicator indicative of at least one clarity in which to at least one of attend, engage, or participate by the individual, the at least one clarity including at least one spiritual clarity, mental clarity, or physical clarity;
receiving, by the at least one recovery server, at least one support contact indicator, the at least one support contact indicator indicative of at least one identifier or contact information of at least one supporter of the individual;
creating, by the at least one recovery server, at least one individual profile of the individual based at least in part on at least one of the individual's personal data, the at least one meeting indicator, the at least one clarity indicator, or the at least one support contact indicator;
storing, by the at least one recovery server, the at least one individual profile of the individual in at least one nontransitory computer-readable memory;
determining, by the at least one recovery server, at least one progress threshold based at least in part on the at least one individual profile of the individual;
receiving, by the at least one recovery server, at least one meeting attendance indicator, the at least one meeting attendance indicator indicative of whether the individual at least one of attends or attended one or more of the at least one meeting of the one or more of the at least one meeting;
in response to receiving the at least one meeting attendance indicator,
determining, by the at least one recovery server, whether the individual at least one of attends or attended the one or more of the at least one meeting;
receiving, by the at least one recovery server, at least one clarity success indicator, the at least one clarity success indicator indicative of whether the individual at least one of attends, participates in, engages in, attended, participated in, or engaged in one or more of the at least one clarity;
in response to receiving the at least one clarity success indicator, determining, by the at least one recovery server, whether the individual at least one of attends, participates in, engages in, attended, participated in, or engaged in the one or more of the at least one clarity of the one or more of the at least one clarity;
receiving, by the at least one recovery server, at least one supporter communication indicator, the at least one supporter communication indicator indicative of whether the individual at least one of communicates or communicated with one or more of the at least one supporter of the individual;
in response to receiving the at least one supporter communication indicator, determining, by the at least one recovery server, whether the individual at least one of communicates or communicated with the one or more of the at least one supporter of the individual;
generating, by the at least one recovery server, at least one accountability score of the individual based at least in part on i) whether the individual at least one of attends or attended the one or more of the at least one meeting, ii) whether the individual at least one of attends, participates in, engages in, attended, participated in, or engaged in the one or more of the at least one clarity, and iii) whether the individual at least one of communicates or communicated with the one or more of the at least one supporter of the individual;
determining, by the at least one recovery server, whether one or more of the accountability score of the individual at least one of equals or exceeds one or more of the at least one progress threshold;
in response to the one or more of the at least one accountability score of the individual at least one of equaling or exceeding the one or more of the at least one progress threshold, providing, by the at least one recovery server, at least one progress success indicator to at least one respective personal communication device of at least one of the individual or the at least one supporter of the individual, each of the at least one respective personal communication devices having at least one respective display, the at least one progress success indicator causing the at least one respective personal communication device to display, via the at least one respective display, at least one progress success notification that indicates to at least one of the individual or the at least one supporter of the individual that the one or more of the at least one accountability score of the individual at least one of equals or exceeds the one or more of the at least one progress threshold; and
in response to the one or more of the at least one accountability score of the individual failing to at least one of equal or exceed the one or more of the at least one progress threshold, providing, by the at least one recovery server, at least one progress support indicator to the at least one respective personal communication device of at least one of the individual or the at least one supporter of the individual, the at least one progress support indicator causing the at least one respective personal communication device to display via the at least one respective display at least one progress support alert that alerts at least one of the individual or the at least one supporter of the individual that the one or more of the at least one accountability score of the individual fails to at least one of equal or exceed the one or more of the at least one progress threshold.

2. The method of claim 1 wherein receiving an individual's personal data further includes:
receiving, by the at least one recovery server, at least one sobriety birthday indicator, the at least one sobriety birthday indicator indicative of at least one sobriety birthday of the individual, the at least one sobriety birthday defined by at least one of i) at least one respective date that the individual most recently engaged or participated in at least one activity or behavior or ii) at least one respective date that the individual first began a current duration of abstinence from the at least one activity or behavior, the at least one activity or behavior being at least one to which the at least one individual has at least one addiction; and
receiving, by the at least one recovery server, at least one support routine indicator, the at least one support routine indicator indicative of at least one course of treatment of addiction of the individual to at least one activity, behavior, substance, or item, the at least one course of treatment including routine attendance of the at least one meeting.

3. The method of any of claim 1, further comprising:
determining, by the at least one recovery server, at least one scheduled respective date, respective time, and respective location of each of the at least one meeting,
wherein receiving at least one meeting attendance indicator further includes:
receiving, via at least one respective geolocation system that communicatively couples to the at least one respective personal communication device of the individual and by the at least one recovery server, at least one location indicator, the at least one location indicator indicative of at least one estimated geographic location of the at least one respective personal communication device of the individual as estimated by the at least one respective geolocation system that communicatively couples to the at least one respective personal communication device of the individual; and
wherein determining whether the individual at least one of attends or attended the one or more of the at least one meeting further includes:
determining, by the at least one recovery server, whether the at least one estimated geographic location of the at least one respective personal communication device of the individual at least one of coincides or coincided with the at least one respective location of the one or more of the at least one meeting at the at least one respective date and respective time of the one or more of the at least one meeting.

4. The method of claim 3 wherein receiving at least one location indicator further includes:
determining, by the at least one recovery server, whether the at least one respective geolocation system that communicatively couples to the at least one respective personal communication device of the individual is enabled; and
in response to the at least one respective geolocation system that communicatively couples to the at least one respective personal communication device of the individual not being enabled, providing, to the at least one respective personal communication device of the individual and by the at least one recovery server, at least one geolocation enablement communication that causes the at least one respective personal communication device of the individual to display via the respective display at least one geolocation enablement notification that at least one of instructs or prompts the individual to enable the at least one geolocation system that communicatively couples to the at least one respective personal communication device of the individual.

5. The method of any of claim 1, further comprising:
determining, by the at least one recovery server, at least one respective clarity biometric value of each of the at least one clarity, the at least one respective clarity biometric value indicative of at least one estimated respective biometric of the individual while the individual at least one of attends, participates in, or engages in each of the at least one clarity,
wherein receiving at least one clarity success indicator further includes:
receiving, via at least one respective biometric sensor that communicatively couples to the at least one respective personal communication device of the individual and by the at least one recovery server, at least one biometric indicator, the at least one biometric indicator indicative of at least one estimated biometric of the individual as estimated by the at least one respective biometric sensor that communicatively couples to the at least one respective personal communication device of the individual; and
wherein determining whether the individual at least one of attends, participates in, engages in, attended, participated in, or engaged in the one or more of the at least one clarity further includes:
determining, by the at least one recovery server, whether the at least one estimated biometric of the individual coincides or coincided with the at least one estimated respective biometric of the individual while the individual attends, participates in, or engages in the one or more of the at least one clarity.

6. The method of claim 5 wherein determining at least one respective clarity biometric value of each of the at least one clarity further includes:
determining, by the at least one recovery server, the at least one respective clarity biometric value of each of the at least one clarity based at least in part on at least one of i) at least one historical estimated respective biometric of the individual while the individual attends, participates in, or engages in each of the at least one clarity or ii) at least one portion of the at least one individual profile of the individual.

7. The method of any of claim 5 wherein the at least one spiritual clarity includes at least one of prayer, meditation, or religious service,
wherein the at least one mental clarity includes at least one of creative exercise, reading, or writing, and
wherein the at least one physical clarity includes at least one of physical exercise, hiking, or sleeping.

8. A system, comprising:
at least one personal communication device of an individual, the at least one personal communication device of the individual having at least one respective processor, nontransitory computer-readable memory communicatively coupled to the at least one respective processor, display communicatively coupled to the at least one respective processor, and individual input interface communicatively coupled to the at least one respective processor, the individual having at least one addiction to at least one activity, behavior, item, or substance; and
at least one recovery server that communicatively couples to the personal communication device of the individual via at least one communications network, the at least one recovery server having at least one processor and nontransitory computer-readable memory communicatively coupled to the at least one processor,
wherein one or more of the at least one respective nontransitory computer-readable memory of the at least one personal communication device of the individual or the at least one nontransitory computer-readable memory of the at least one recovery server having at least one of respective processor-executable instructions or data stored therein, the at least one of respective processor-executable instructions or data which, when executed by one or more of the at least one respective processor of the at least one personal communication device or the at least one processor of the at least one recovery server, cause the at least one personal communication device of the individual and the at least one recovery server to cooperatively support recovery of the individual by one or more of the at least one personal communication device of the individual or the at least one recovery server:
obtaining personal data of the individual;
obtaining at least one of each indicator in a group of indicators that includes two or more selected from a meeting indicator, a clarity indicator, or a support contact indicator, the at least one meeting indicator indicative of at least one meeting to attend by the individual, the at least one meeting including at least one support meeting, group session, or one-on-one session, the at least one clarity indicator indicative of at least one clarity in which to at least one of attend, engage, or participate by the individual, the at least one clarity including at least one spiritual clarity, mental clarity, or physical clarity, the at least one support contact indicator indicative of at least one identifier or contact information of at least one supporter of the individual;
creating at least one individual profile of the individual based at least in part on at least one of the individual's personal data, the at least one meeting indicator, the at least one clarity indicator, or the at least one support contact indicator;
storing the at least one individual profile of the individual in one or more of the at least one respective nontransitory computer-readable memory of the at least one personal communication device or the at least one nontransitory computer-readable memory of the at least one recovery server;
determining at least one progress threshold based at least in part on the at least one individual profile of the individual;
obtaining at least one of each respective indicator in an indicator group of indicators that includes two or more selected from meeting attendance indicator, clarity success indicator, or supporter communication indicator, the at least one meeting attendance indicator indicative of whether the individual at least one of attends or attended one or more of the at least one meeting of the one or more of the at least one meeting, the at least one clarity success indicator indicative of whether the individual at least one of attends, participates in, engages in, attended, participated in, or engaged in one or more of the at least one clarity, the at least one supporter communication indicator indicative of whether the individual at least one of communicates or communicated with one or more of the at least one supporter of the individual;
in response to obtaining the at least one meeting attendance indicator, determining whether the individual at least one of attends or attended the one or more of the at least one meeting;
in response to obtaining the at least one clarity success indicator, determining whether the individual at least one of attends, participates in, engages in, attended, participated in, or engaged in the one or more of the at least one clarity of the one or more of the at least one clarity;
in response to obtaining the at least one supporter communication indicator, determining whether the individual at least one of communicates or communicated with the one or more of the at least one supporter of the individual;
generating at least one accountability score of the individual based at least in part on at least two of i) whether the individual at least one of attends or attended the one or more of the at least one meeting, ii) whether the individual at least one of attends, participates in, engages in, attended, participated in, or engaged in the one or more of the at least one clarity, or iii) whether the individual at least one of communicates or communicated with the one or more of the at least one supporter of the individual;
determining whether one or more of the accountability score of the individual at least one of equals or exceeds one or more of the at least one progress threshold;
in response to the one or more of the at least one accountability score of the individual at least one of equaling or exceeding the one or more of the at least one progress threshold, providing at least one progress success indicator to one or more of the at least one personal communication device of the individual or at one personal communication device of the at least one supporter of the individual, the at least one personal communication device of the at least one supporter of the individual having at least one respective display, the at least one progress success indicator causing the one or more of the at least one personal communication device of the individual or the at least one personal communication device of the at least one supporter of the individual to display, via the at least one respective display of the one or more of the at least one personal communication device of the individual or the at least one personal communication device of the at least one supporter of the individual, at least one progress success notification that indicates to at least one of the individual or the at least one supporter of the individual that the one or more of the at least one accountability score of the individual at least one of equals or exceeds the one or more of the at least one progress threshold; and
in response to the one or more of the at least one accountability score of the individual failing to at least one of equal or exceed the one or more of the at least one progress threshold, providing at least one progress support indicator to the one or more of the at least one personal communication device of the individual or the at least one personal communication device of the at least one supporter of the individual, the at least one progress support indicator causing the one or more of the at least one personal communication device of the individual or the at least one personal communication device of the at least one supporter of the individual to display, via the at least one respective display of the one or more of the at least one personal communication device of the individual or the at least one personal communication device of the at least one supporter of the individual, at least one progress support alert that alerts at least one of the individual or the at least one supporter of the individual that the one or more of the at least one accountability score of the individual fails to at least one of equal or exceed the one or more of the at least one progress threshold.

9. The system of claim 8 wherein obtaining an individual's personal data further includes:
obtaining at least one sobriety birthday indicator, the at least one sobriety birthday indicator indicative of at least one sobriety birthday of the individual, the at least one sobriety birthday defined by at least one of i) at least one respective date that the individual most recently engaged or participated in at least one activity or behavior or ii) at least one respective date that the individual first began a current duration of abstinence from the at least one activity or behavior, the at least one activity or behavior being at least one to which the at least one individual has at least one addiction; and
obtaining at least one support routine indicator, the at least one support routine indicator indicative of at least one course of treatment of addiction of the individual to at least one activity, behavior, substance, or item, the at least one course of treatment including routine attendance of the at least one meeting.

10. The system of any of claim 8 wherein the at least one of respective processor-executable instructions or data which, when executed by one or more of the at least one respective processor of the at least one personal communication device of the individual or the at least one processor of the at least one recovery server, further cause the at least one personal communication device of the individual and the at least one recovery server to cooperatively support recovery of the individual by one or more of the at least one personal communication device of the individual or the at least one recovery server:
determining at least one scheduled respective date, respective time, and respective location of each of the at least one meeting,
wherein the indicator group includes the at least one meeting indicator,
wherein obtaining at least one of each respective indicator in an indicator group of indicators that includes two or more selected from meeting attendance indicator, clarity success indicator, or supporter communication indicator further includes:
obtaining, via at least one respective geolocation system that communicatively couples to the at least one personal communication device of the individual, at least one location indicator, the at least one location indicator indicative of at least one estimated geographic location of the at least one personal communication device of the individual as estimated by the at least one respective geolocation system that communicatively couples to the at least one personal communication device of the individual; and
wherein determining whether the individual at least one of attends or attended the one or more of the at least one meeting further includes:
determining whether the at least one estimated geographic location of the at least one personal communication device of the individual at least one of coincides or coincided with the at least one respective location of the one or more of the at least one meeting at the at least one respective date and respective time of the one or more of the at least one meeting.

11. The system of claim 10 wherein obtaining at least one location indicator further includes:
determining whether the at least one respective geolocation system that communicatively couples to the at least one personal communication device of the individual is enabled; and
in response to the at least one respective geolocation system that communicatively couples to the at least one personal communication device of the individual not being enabled, displaying, via the respective display of the at least one personal communication device of the individual, at least one geolocation enablement notification that at least one of instructs or prompts the individual to enable the at least one geolocation system that communicatively couples to the at least one personal communication device of the individual.

12. The system of any of claim 8 wherein the at least one of respective processor-executable instructions or data which, when executed by one or more of the at least one respective processor of the at least one personal communication device of the individual or the at least one processor of the at least one recovery server, further cause the at least one personal communication device of the individual and the at least one recovery server to cooperatively support recovery of the individual by one or more of the at least one personal communication device of the individual or the at least one recovery server:
determining at least one respective clarity biometric value of each of the at least one clarity, the at least one respective clarity biometric value indicative of at least one estimated respective biometric of the individual while the individual at least one of attends, participates in, or engages in each of the at least one clarity,
wherein the indicator group includes the at least one clarity indicator,
wherein obtaining at least one clarity success indicator further includes:
obtaining, via at least one respective biometric sensor that communicatively couples to the at least one personal communication device of the individual, at least one biometric indicator, the at least one biometric indicator indicative of at least one estimated biometric of the individual as estimated by the at least one respective biometric sensor that communicatively couples to the at least one personal communication device of the individual; and
wherein determining whether the individual at least one of attends, participates in, engages in, attended, participated in, or engaged in the one or more of the at least one clarity further includes:
determining whether the at least one estimated biometric of the individual coincides or coincided with the at least one estimated respective biometric of the individual while the individual attends, participates in, or engages in the one or more of the at least one clarity.

13. The system of claim 12 wherein determining at least one respective clarity biometric value of each of the at least one clarity further includes:
determining the at least one respective clarity biometric value of each of the at least one clarity based at least in part on at least one of i) at least one historical estimated respective biometric of the individual while the individual attends, participates in, or engages in each of the at least one clarity or ii) at least one portion of the at least one individual profile of the individual.

14. The system of any of claim 8 wherein the indicator group includes the at least one support contact indicator, and
wherein determining whether the individual at least one of communicates or communicated with the one or more of the at least one supporter of the individual further includes:
analyzing at least one telephone call, text message, or email that the at least one personal communication device of the individual at least one of transmits or receives; and
determining whether the at least one analyzed telephone call, text message, or email includes the at least one supporter of the individual as at least one recipient or sender of the at least one analyzed telephone call, text message, or email.

15. The system of any of claim 8 wherein the indicator group includes the at least one support contact indicator, and
wherein determining whether the individual at least one of communicates or communicated with the one or more of the at least one supporter of the individual further includes:
obtaining, via at least one respective geolocation system that communicatively couples to the at least one personal communication device of the individual, at least one individual location indicator, the at least one individual location indicator indicative of at least one estimated individual geographic location of the at least one personal communication device of the individual as estimated by the at least one respective geolocation system that communicatively couples to the at least one personal communication device of the individual;
obtaining, via at least one respective geolocation system that communicatively couples to the at least one personal communication device of the at least one supporter of the individual, at least one supporter location indicator, the at least one supporter location indicator indicative of at least one estimated supporter geographic location of the at least one personal communication device of the at least one supporter of the individual as estimated by the at least one respective geolocation system that communicatively couples to the at least one personal communication device of the at least one supporter of the individual; and
determining whether the at least one estimated individual geographic location of the at least one personal communication device of the individual at least one of coincides or coincided with the at least one estimated supporter geographic location of the at least one personal communication device of the at least one supporter of the individual.

16. The system of any of claim 8 wherein the indicator group includes the at least one support contact indicator, and
wherein determining whether the individual at least one of communicates or communicated with the one or more of the at least one supporter of the individual further includes:
obtaining, via at least one respective near field communication (NFC) system or personal area network (PAN) system that communicatively couples to the at least one personal communication device of the individual, at least one NFC or PAN communication indicator, the at least one NFC or PAN communication indicator indicative of at least one identifier of at least one NFC system, PAN system, or personal communication device of another to which the NFC or PAN system that communicatively couples to the at least one personal communication device of the individual communicatively couples; and
determining whether the at least one identifier of the at least one NFC system, PAN system, or personal communication device of the other matches at least one identifier of at least one NFC system, PAN system, or personal communication device of the at least one supporter of the individual.

17. The system of any of claim 8 wherein the at least one of respective processor-executable instructions or data which, when executed by one or more of the at least one respective processor of the at least one personal communication device of the individual or the at least one processor of the at least one recovery server, further cause the at least one personal communication device of the individual and the at least one recovery server to cooperatively support recovery of the individual by one or more of the at least one personal communication device of the individual or the at least one recovery server:
intermittently or continuously adjusting the one or more of the at least one progress threshold based at least in part on at least one of a number or percentage of scheduled activities of the individual that the individual successfully achieves in a defined period of time that includes multiple scheduled activities.

18. The system of any of claim 8 wherein the at least one of respective processor-executable instructions or data which, when executed by one or more of the at least one respective processor of the at least one personal communication device of the individual or the at least one processor of the at least one recovery server, further cause the at least one personal communication device of the individual and the at least one recovery server to cooperatively support recovery of the individual by one or more of the at least one personal communication device of the individual or the at least one recovery server:
obtaining, via at least one respective biometric sensor that communicatively couples to the at least one personal communication device of the individual, at least one biometric indicator, the at least one biometric indicator indicative of at least one estimated biometric of the individual as estimated by the at least one respective biometric sensor that communicatively couples to the at least one personal communication device of the individual;
determining at least one stress level that the at least one estimated biometric of the individual indicates; and
intermittently or continuously adjusting at least one of respective frequency or respective duration of at least one scheduled activity of the individual responsive to the stress level.
